(19) United States
(12) Patent Application Publication
(10) Pub. No.: US 2002/0098190 A1
(43) Pub. Date: Jul. 25, 2002

(54) Compositions and methods for treating tumors bearing HMFG and CEA antigens

(76) Inventors: Malaya Chatterjee, Fort Wright, KY (US); Kenneth A. Foon, Fermont, CA (US)

Correspondence Address: Catherine M. Polizzi, Morrison & Foerster LLP, 755 Page Mill Road, Palo Alto, CA, 94304-1018, US

(21) Appl. No.: 09/861,294

(22) Filed: May 17, 2001

Related U.S. Application Data
(63) Continuation-in-part of application No. 09/096,244, filed on Jun. 11, 1998.
(60) Provisional application No. 60/049,540, filed on Jun. 13, 1997.

(51) Int. Cl. A61K 39/395; C12N 5/06

(52) U.S. Cl. 424/155.1; 435/344

(57) ABSTRACT

The present invention provides methods and compositions for treating HMFG- and CEA-associated tumors using the anti-idiotype antibody 11D10 in conjunction with anti-idiotype antibody 3H1.

### CROSS-REFERENCE TO RELATED APPLICATIONS

This application claims the benefit of U.S. application Ser. No. 09/096,244, filed Jun. 11, 1998, which claims the benefit of provisional application 60/049,540, filed Jun. 13, 1997, which are incorporated in their entirety herein by reference.

### STATEMENT OF RIGHTS TO INVENTIONS MADE UNDER FEDERALLY SPONSORED RESEARCH

This research is sponsored by the following government grants: National Cancer Institute (NCI) Program Grant U01-CA-65748; National Institutes of Health NIH R01 CA-60000. The government may have certain rights in this invention.

### TECHNICAL FIELD

This invention relates to uses of anti-idiotype antibodies. More particularly, it relates to methods of treatment using anti-idiotype antibody 11D10, or anti-idiotype antibody 11D10 in conjunction with anti-idiotype antibody 3H1.

### BACKGROUND ART

In spite of extensive medical research and numerous advances, cancer remains the second leading cause of death in the United States. Breast cancer is the most common cause of cancer deaths in women with over 150,000 new cases diagnosed annually. Colorectal cancer is the third most common cancer and the second leading cause of cancer deaths. While the traditional modes of therapy, such as surgery, radiotherapy and chemotherapy, are widely used and are in many instances successful, the still existing high death rate from cancers such as breast compels the need for alternative or additional modes of therapy.

Even if a patient responds to traditional modes of therapy, there is often a significant risk of recurrence of the disease. This is especially true if the disease has spread when diagnosed. Even after “successful” treatment, in which a remission is observed, a patient can have high risk of recurrence, and can only “watch and wait.” There are presently no further courses of action to delay or prevent recurrence.

One approach to cancer therapy has been immunotherapy. However, immunotherapy of human cancer using tumor cells or tumor-derived vaccines has been disappointing for several reasons. It has been consistently difficult to obtain large quantities or purified tumor-associated antigens which are often chemically ill-defined and difficult to purify. In addition, there remains the problem of immunobiological response potential against tumor antigens, or in other words, the question of whether a cancer patient can effectively mount an immune response against his or her tumor. Tumor-associated antigens (TAA) are often a part of “self” and usually evoke a very poor immune response in a tumor-bearing host due to tolerance to the antigens, such as T cell-mediated suppression. Moreover, cancer patients tend to be immunosuppressed and only respond to certain T-dependent antigens.

Immunobiologists have learned that a poor antigen (in terms of eliciting an immune response) can be turned into a strong antigen by changing the molecular environment. Changes of hapten carrier allow T cell helper cells to become active, making the overall immune response stronger. Thus, changing the carrier can also turn a tolerogenic antigen into an effective antigen. McBride et al. (1986) Br. J. Cancer 53:707. Often the immunological status of a cancer patient is suppressed such that the patient is only able to respond to certain T-dependent antigens and not to other antigen forms. From these considerations, it would make sense to introduce molecular changes into the tumor associated antigens before using them as vaccines. Unfortunately, this is impossible to accomplish for most tumor antigens, because they are not well defined and are very hard to purify.

The network hypothesis of Lindemann ((1973) Ann. Immunol. 124:171-184) and Jerne ((1974) Ann. Immunol. 125:373-389) offers an elegant approach to transform epitope structures into idiotypic determinants expressed on the surface of antibodies. According to the network concept, immunization with a given tumor-associated antigen will generate production of antibodies against this tumor-associated antigen, termed Ab1; this Ab1 is then used to generate a series of anti-idiotype antibodies against the Ab1, termed Ab2. Some of these Ab2 molecules can effectively mimic the three-dimensional structure of the tumor-associated antigen identified by the Ab1. These particular anti-idiotypes called Ab2β fit into the paratopes of Ab1, and express the internal image of the tumor-associated antigen. The Ab2β can induce specific immune responses similar to those induced by the original tumor-associated antigen and can, therefore, be used as surrogate tumor-associated antigens. Immunization with Ab2β can lead to the generation of anti-anti-idiotype antibodies (Ab3) that recognize the corresponding original tumor-associated antigen identified by Ab1. Because of this Ab1-like reactivity, the Ab3 is also called Ab1′ to indicate that it might differ in its other idiotypes from Ab1.

A potentially promising approach to cancer treatment is immunotherapy employing anti-idiotype antibodies. In this form of therapy, an antibody mimicking an epitope of a tumor-associated protein is administered in an effort to stimulate the patient's immune system against the tumor, via the tumor-associated protein. WO 91/11465 describes methods of stimulating an immune response in a human against malignant cells or an infectious agent using primate anti-idiotype antibodies. However, not all anti-idiotype antibodies can be used in therapeutic regimens against tumors. First, only a fraction of antibodies raised against an Ab1 are limited in their reactivity to the paratope of Ab1 (i.e., are non-reactive against features shared with other potential antibodies in the host). Second, anti-idiotype antibodies are not necessarily immunogenic. Third, even if an anti-idiotype elicits an immune response, only a fraction of these immunogenic anti-idiotypes elicit an immune response against the tumor antigen and not against other antigens with less specificity. Moreover, since different cancers have widely varying molecular and clinical characteristics, it has been suggested that anti-idiotype therapy should be evaluated on a case by case basis, in terms of tumor origin and antigens expressed.

Anti-Id monoclonal antibodies structurally resembling tumor-associated antigens have been used as antigen substitutes in cancer patients. Herlyn et al. (1987) Proc. Natl. Acad. Sci. U.S.A. 84:8055-8059; Mittleman et al. (1992) Proc. Natl. Acad. Sci. U.S.A. 89:466-470; Chatterjee et al. (1993) Ann. N.Y. Acad. Sci. 690:376-278. All of these studies were conducted with patients having advanced disease. Based on the observed immune response in at least some of the patients, it has been proposed that the anti-Id provides a partial analog of the tumor-associated antigen in an immunogenic context.

Human milk fat globules (HMFG) are milk fat globules secreted into breast milk by the breast epithelial cell, and are composed of fat droplets enveloped by plasma membrane. As such, HMFG is a rich source of epithelial membrane-associated antigens. One antigen component of HMFG is a high molecular weight, membrane-associated mucin that is associated with breast and other cancers such as ovarian, lung, and pancreas. The mucin contains a protein with known amino acid sequences derived from the cDNA. Semipurified HMFG is available in small quantities from several sources and can be used in serological assays. Peterson et al. (1990) Hybridoma 9:221-235. However, HMFG is extremely difficult to isolate and purify, and purified HMFG is not available for patient immunization or animal studies. Further, inasmuch as some of the epitopes on HMFG are shared by normal tissues, specifically by nonpenetrating glycoproteins, immunization with intact HMFG molecule might trigger potentially harmful autoimmune reactions. An immune reaction against a tumor-associated epitope, on the other hand, would be much more desirable.

A series of murine monoclonal antibodies (mAbs) that recognize components of HMFG have been described that are primarily associated with human breast carcinomas and not with most normal tissues. Chatterjee et al. (1993) Ann. N.Y. Acad. Sci. 690:376-377; Ceriani et al. (1983) Somatic Cell Genet. 9:415-427. Among these mAbs, MC-10 (BrE-1) is the most restricted and specific, reacting with a large molecular weight (MW, 400,000) mucin-like protein present at high density and on >80% breast cancer cells and minimally expressed on a few normal tissues, such as the epithelial lining of lung and kidney tubules. Ceriani et al. (1983); Ceriani et al. (1990) Antibody Immunoconjugates and Radiopharmaceuticals 3:181-198.

Carcinoembryonic antigen (CEA) is a 180,000-kiloDalton glycoprotein tumor-associated antigen present on endodermally-derived neoplasms of the gastrointestinal tract, such as colorectal and pancreatic cancer, as well as other adenocarcinomas such as breast and lung cancers. CEA is also found in the digestive organs of the human fetus. Circulating CEA can be detected in the great majority of patients with CEA-positive tumors. Specific monoclonal antibodies have been raised against CEA and some have been radiolabeled for diagnostic and clinical studies. Hansen et al. (1993) Cancer 71:3478-3485; Karoki et al. (1992) Hybridoma 11:391-407; Goldenberg (1993) Am. J. Med. 94:297-312. As with most tumor-associated antigens which are seen as self-antigens by the immune system, cancer patients are immunologically “tolerant” to CEA, possibly due to its oncofetal origin. Studies to date on patients with CEA-positive tumors have not demonstrated the ability to generate immunity to CEA. Thus, immunotherapy based on CEA has heretofore not been possible.

CEA nonetheless is an excellent tumor-associated antigen for active immunotherapy with anti-idiotype antibody for several reasons. CEA is typically present at high levels on the tumor cell surface. CEA is also one of the most well-characterized antigens, as its gene sequence is known and its three dimensional structures have been identified. CEA is a member of the immunoglobulin supergene family located on chromosome 19 which is thought to be involved in cell-cell interactions.

Inasmuch as some of the epitopes on CEA are shared by normal tissues, immunization with intact CEA molecule might trigger potentially harmful autoimmune reactions. An immune reaction against a tumor-associated epitope, on the other hand, would be desirable. An appropriate anti-idiotype antibody would be an excellent candidate to induce anti-tumor immunity in CEA positive cancer patients. A number of investigators have generated anti-idiotype antibodies in rats, mice, baboons and humans that mimic CEA. See, e.g., Hinoda et al. (1995) Tumor Biol. 16:48-55; Losman et al. (1994) Int. J. Cancer 56:580-584; Irvine et al. (1993) Cancer Immunol. Immunother. 36:281-292. However, given the size of CEA (and likely numerous epitopes), and the fact that CEA is expressed on some normal tissues, it was not known whether anti-idiotype antibodies would be effective in eliciting an anti-CEA response that effects anti-tumor immunity.

For other discussions regarding anti-idiotype antibodies in the cancer context, see also generally Foon & Bhattacharya-Chatterjee, Anti-idiotype antibodies: Novel therapeutic approach to cancer therapy in: Tumor Immunology and Cancer Therapy (1994), p. 281-292 (Goldfarb & Whiteside, eds.); Bhattacharya-Chatterjee et al., Cancer Immunol. Immunother. (2000), 49:133-141; Bhattacharya-Chattejee et al., Immunology Letters (2000), 74:51-58; Lewin, Science (1987), 237:1570; Reeck et al., Cell (1987), 50:667; Chatterjee et al., Cancer Immuno. Immunother. (1994), 38:75-82; Rudikoff et al., Proc. Natl. Acad. Sci. USA (1982), 79:1979; Panka et al., Proc. Natl. Acad. Sci. USA (1988), 85:3080-3084; Amit et al., Science (1986), 233:747-753; Chatterjee et al., J. Immunol. (1988), 141:1398-1403; Browning et al., Cell (1993), 72:847-856; Mo et al., Eur. J. Immuno. (1993), 23:2503-2510; Liu et al., J. immunol. (1987), 139:3521-3526; Foon et al., J. Clin. Invest. (1995), 96:334-342; Hinoda et al., Tumor biol. (1995), 16:48-55; Herlyn et al., Proc. Natl. Acad. Sci. (1987), 84:8055-8059; Mittelman et al., Cancer Res. (1994), 54:415-421; Erlichman et al., J. Clin. Oncol. (1988), 6:469-475; Johnson et al., Cancer Treatment Review (1975), 2:1-31; U.S. Pat. Nos. 5,808,033; 5,334,708 and 5,840,299; PCT Pub. Nos. WO 97/22699, 98/56419, 96/20277, 96/02019, 97/38725, 91/09967.

Carcinomas of the gastrointestinal tract and recurrent breast cancer are often not curable by standard therapies. Even if a patient responds to traditional therapy, there is often a significant risk of recurrence. Thus, new therapeutic approaches for these diseases are needed. The present invention overcomes deficinecies in the prior art by providing a monoclonal anti-idiotype antibody (11D10) as an antigen (Ag) that elicits an immune response against HMFG. The present invention also overcomes the deficiencies in the prior art by providing methods of treatment for HMFG and CEA-associated tumors using a combination of anti-idiotype antibodies which escape immune tolerance and induce an anti-HMFG and anti-CEA immune responses.

All references cited herein are incorporated by reference in their entirety.

### DISCLOSURE OF THE INVENTION

The present invention is directed to therapeutic uses of the anti-idiotype antibody 11D10. The present invention is also directed to using anti-idiotypic antibodies for HMFG and CEA (i.e., based on HMFG and CEA) to treat HMFG- and CEA-associated disease states, espcially HMFG- and CEA-associated tumors (tumors bearing HMFG and CEA). The invention also provides compositions comprising a combination of these anti-idiotypic antibodies.

Accordingly, one aspect of the invention is methods of delaying development of HMFG-associated tumors in an individual having a low tumor burden, particularly high risk individuals. These methods include administration of an effective amount of anti-idiotype antibody 11D10 to the individual. In another aspect, the invention further includes administration of 11D10 with an adjuvant.

In another aspect, methods are provided for treatment of an HMFG-associated tumor in an individual with a low tumor burden which entail administering an effective amount of 11D10 to the individual.

In one aspect, the invention provides methods of treating (which can include delaying development and/or recurrence of) an HMFG- and CEA-associated tumor in an individual, comprising administering an effective amount of an anti-idiotype antibody for HMFG in conjunction with an effective amount of an anti-idiotype antibody for CEA. In one embodiment, the method comprises administering an effective amount of a first antibody comprising the light and heavy chain variable region sequences contained in SEQ ID NO:2 and SEQ ID NO:4, respectively, and a second antibody comprising the light and heavy chain variable region sequences contained in SEQ ID NO:6 and SEQ ID NO:8, respectively, to the individual, wherein said administration results in treatment of (which can include delaying development and/or recurrence of) the HMFG- and CEA-associated tumor in the individual. In some embodiments, said first antibody is antibody 11D10, which is produced by a hybridoma cell line deposited at the American Type Culture Collection (ATCC) as Accession No. 12020, or progeny thereof. In yet another embodiment, said second antibody is antibody 3H1, which is produced by a hybridoma cell line deposited at the American Type Culture Collection (ATCC) as Accession No. HB12003, or progeny thereof. In still another embodiment, said first antibody is antibody 11D10, which is produced by a hybridoma cell line deposited at the American Type Culture Collection (ATCC) as Accession No. 12020, or progeny thereof, and the second antibody is antibody 3H1, which is produced by a hybridoma cell line deposited at the American Type Culture Collection (ATCC) as Accession No. HB12003, or progeny thereof.

In certain embodiments, the methods of the invention comprise administering to the treated individual a first polypeptide having immunological activity of anti-idiotype antibody 11D10 in conjunction with a second polypeptide having immunological activity of anti-idiotype antibody 3H1, wherein the first polypeptide comprises an immunoglobulin variable region containing at least one (preferably 1, 2 or 3) light chain complementarity determining regions (CDRs) of antibody 11D10, and/or an immunoglobulin variable region containing at least one (preferably 1, 2 or 3) heavy chain CDRs of antibody 11D10, wherein the second polypeptide comprises an immunoglobulin variable region containing at least one (preferably 1, 2 or 3) light chain complementarity determining regions (CDRs) of antibody 3H1, and/or an immunoglobulin variable region containing at least one (preferably 1, 2 or 3) heavy chain CDRs of antibody 3H1, and wherein the immunological activity of the first poplypeptide is an ability to stimulate a specific immune response against HMFG and the the immunological activity of the second poplypeptide is an ability to stimulate a specific immune response against CEA. In some of these embodiments, the first polypeptide is antibody 11D10. In some embodiments, the second polypeptide is antibody 3H1. In some embodiments, the first polypeptide is antibody 11D10 and the second polypeptide is antibody 3H1.

In some embodiments of the methods of the invention, the individual who is treated is high risk. In certain embodiments, the individual is in the adjuvant setting. In other embodiments, the individual has a low tumor burden for HMFG- and CEA-associated tumor(s).

In still other embodiments of the methods of the invention, either or both of the antibodies (or polypeptides) are administered with an adjuvant. The adjuvant can be any known in the art, preferably aluminum hydroxide.

In some embodiments, methods of the invention are conduction in conjunction with other forms of therapy, including, for example, chemotherapy, hormonal therapy (generally first-line hormonal therapy) and radiation therapy (for example, in patients in an adjuvant setting).

The methods of the invention can be used to treat any HMFG- and CEA-associated tumor. The HMFG- and CEA-associated tumor is preferably of gastrointestinal origin (which is preferably colorectal), of the lung (which is preferably non-small cell lung carcinoma and small cell lung carcinoma), of ovarian origin, or of the breast.

In some embodiments of the methods of the invention, the first antibody and the second antibody are each administered in an amount of about 1 mg to about 4 mg. In other embodiments, the first antibody and the second antibody are each administered in an amount of about 2 mg. The first antibody and the second antibody can each be administered at weekly intervals, every two weeks or monthly. The first antibody and the second antibody can be heat-treated prior to administration.

In some embodiments, the treated individual has a circulating CEA level of less than about 50 ng/ml. In some embodiments, the treated individual is human.

In another aspect, the invention provides methods of identifying an individual suitable for treatment using the methods of the invention, said methods comprising detecting both HMFG and CEA in or on the cells of the same tumor in an individual, whereby the presence of HMFG and CEA is indicative of an individual suitable for treatment by the treatment methods of the invention. In some embodiments, the presence of HMFG is detected using an immunoreagent such as an antibody that is immunospecific for HMFG (i.e., specifically binds HMFG), and the presence of CEA is detected using an immunoreagent such as an antibody that is immunospecific for CEA (i.e., specifically binds CEA). Preferably, the immunoreagent for HMFG is immunospecific for an epitope against which anti-idiotype antibody 11D10 is capable of raising an immune response, and the immunoreagent for CEA is immunospecific for an epitope against which anti-idiotype antibody 3H1 is capable of raising an immune response. In other embodiments, the presence of HMFG and CEA is detected by analysis of transcription products of genes encoding an antigen for HMFG and CEA, respectively.

In another aspect, the invention provides methods of delaying development of an HMFG- and CEA-associated tumor comprising: (a) identifying a suitable individual for using methods of the invention to delay development of HMFG- and CEA-associated tumor; and (b) administering an effective amount of a first antibody comprising the light and heavy chain variable region sequences contained in SEQ ID NO:2 and SEQ ID NO:4, respectively, and a second antibody comprising the light and heavy chain variable region sequences contained in SEQ ID NO:6 and SEQ ID NO:8, respectively, to the individual. In some embodiments, said first antibody is antibody 11D10, which is produced by a hybridoma cell line deposited at the American Type Culture Collection (ATCC) as Accession No. 12020, or progeny thereof. In yet another embodiment, said second antibody is antibody 3H1, which is produced by a hybridoma cell line deposited at the American Type Culture Collection (ATCC) as Accession No. HB12003, or progeny thereof. In still another embodiment, said first antibody is antibody 11D10, which is produced by a hybridoma cell line deposited at the American Type Culture Collection (ATCC) as Accession No. 12020, or progeny thereof, and the second antibody is antibody 3H1, which is produced by a hybridoma cell line deposited at the American Type Culture Collection (ATCC) as Accession No. HB12003, or progeny thereof. In certain embodiments, step (b) comprises administering to the individual a first polypeptide having immunological activity of anti-idiotype antibody 11D10 in conjunction with a second polypeptide having immunological activity of anti-idiotype antibody 3H1, wherein the first polypeptide comprises an immunoglobulin variable region containing at least one (preferably 1, 2 or 3) light chain complementarity determining regions (CDRs) of antibody 11D10, and/or an immunoglobulin variable region containing at least one (preferably 1, 2 or 3) heavy chain CDRs of antibody 11D10, wherein the second polypeptide comprises an immunoglobulin variable region containing at least one (preferably 1, 2 or 3) light chain complementarity determining regions (CDRs) of antibody 3H1, and/or an immunoglobulin variable region containing at least one (preferably 1, 2 or 3) heavy chain CDRs of antibody 3H1, and wherein the immunological activity of the first poplypeptide is an ability to stimulate a specific immune response against HMFG and the the immunological activity of the second poplypeptide is an ability to stimulate a specific immune response against CEA.

In still another aspect, the invention provides methods of treatment of an HMFG- and CEA-associated tumor comprising: (a) identifying a suitable individual for using methods of the invention to treat HMFG- and CEA-associated tumor; and (b) administering an effective amount of a first antibody comprising the light and heavy chain variable region sequences contained in SEQ ID NO:2 and SEQ ID NO:4, respectively, and a second antibody comprising the light and heavy chain variable region sequences contained in SEQ ID NO:6 and SEQ ID NO:8, respectively, to the individual. In some embodiments, said first antibody is antibody 11D10, which is produced by a hybridoma cell line deposited at the American Type Culture Collection (ATCC) as Accession No. 12020, or progeny thereof. In yet another embodiment, said second antibody is antibody 3H1, which is produced by a hybridoma cell line deposited at the American Type Culture Collection (ATCC) as Accession No. HB12003, or progeny thereof. In still another embodiment, said first antibody is antibody 11D10, which is produced by a hybridoma cell line deposited at the American Type Culture Collection (ATCC) as Accession No. 12020, or progeny thereof, and the second antibody is antibody 3H1, which is produced by a hybridoma cell line deposited at the American Type Culture Collection (ATCC) as Accession No. HB12003, or progeny thereof In certain embodiments, step (b) comprises administering to the individual a first polypeptide having immunological activity of anti-idiotype antibody 11D10 in conjunction with a second polypeptide having immunological activity of anti-idiotype antibody 3H1, wherein the first polypeptide comprises an immunoglobulin variable region containing at least one (preferably 1, 2 or 3) light chain complementarity determining regions (CDRs) of antibody 11D10, and/or an immunoglobulin variable region containing at least one (preferably 1, 2 or 3) heavy chain CDRs of antibody 11D10, wherein the second polypeptide comprises an immunoglobulin variable region containing at least one (preferably 1, 2 or 3) light chain complementarity determining regions (CDRs) of antibody 3H1, and/or an immunoglobulin variable region containing at least one (preferably 1, 2 or 3) heavy chain CDRs of antibody 3H1, and wherein the immunological activity of the first poplypeptide is an ability to stimulate a specific immune response against HMFG and the the immunological activity of the second poplypeptide is an ability to stimulate a specific immune response against CEA.

In yet another aspect, the invention provides compositions comprising a combination of an anti-idiotype antibody for HMFG and an anti-idiotype antibody for CEA. In some embodiments, the anti-idiotype antibody for HMFG comprises the light and heavy chain variable region sequences contained in SEQ ID NO:2 and SEQ ID NO:4, respectively, and the anti-idiotype antibody for CEA comprises the light and heavy chain variable region sequences contained in SEQ ID NO:6 and SEQ ID NO:8, respectively. In some embodiments, the anti-idiotype antibody for HMFG is antibody 11D10, which is produced by a hybridoma cell line deposited at the American Type Culture Collection (ATCC) as Accession No. 12020, or progeny thereof. In still other embodiments, the anti-idiotype antibody for CEA is antibody 3H1, which is produced by a hybridoma cell line deposited at the American Type Culture Collection (ATCC) as Accession No. 12003, or progeny thereof. In some embodiments, the compositions comprise a combination of 11D10 and 3H1.

### BRIEF DESCRIPTION OF THE DRAWINGS

FIG. 1 depicts the cDNA sequence (SEQ ID NO:1) and the amino acid sequence (SEQ ID NO:2) of the light chain variable region of 11D10 and adjoining residues. The CDRs and framework regions are indicated.

FIG. 2 depicts the cDNA sequence (SEQ ID NO:3); and the amino acid sequence (SEQ ID NO:4) of the heavy chain variable region of 11D10 and adjoining residues. The CDRs and framework regions are indicated.

FIGS. 3A and B depict the amino acid sequences of the CDR and framework regions of the light chain.(FIG. 3A) and heavy chain (FIG. 3B) variable region of 11D10.

FIG. 4 depicts the cDNA sequence (SEQ ID NO:5; FIG. 4A) and the amino acid sequence (SEQ ID NO:6; FIG. 4B) of the light chain variable region of 3H1 and adjoining residues.

FIG. 5 depicts the cDNA sequence (SEQ ID NO:7; FIG. 5A) and the amino acid sequence (SEQ ID NO:8; FIG. 5B) of the heavy chain variable region of 3H1 and adjoining residues.

FIG. 6 depicts the amino acid sequences of the light chain variable region (SEQ ID NO:6; FIG. 6A) and the heavy chain variable region (SEQ ID NO:8; FIG. 6B) of 3H1. Each variable region consists of 4 framework regions and 3 CDRs.

### MODES FOR CARRYING OUT THE INVENTION

This invention is based upon an ability of 11D10 to generate an HMFG specific immune response in patients who are at high risk of recurrence of HMFG-associated disease. It is also based upon an ability of 3H1 to generate a CEA specific immune response in patients with CEA-associated disease. Various cancers are known to be associated with or express both HMFG and CEA. We have found that a high percentage of different human cancers, including squamous cell and adenocarcinomas (such as non-small cell lung carcinomas), small cell lung carcinomas, ovarian cancers, breast cancers and colorectal cancers bear both HMFG and CEA. Tumors studied for binding for both BrE1 (a mouse monoclonal antibody directed against HMFG) and antibody 8019 (a mouse monoclonal antibody directed against human CEA) (number positive/total number) include: non-small cell lung carcinoma (16/20), small cell lung carcinoma (8/10), ovarian cancer (5/12), breast cancer (5/5), melanoma (0/19) and colorectal cancer (11/12). We believe that administration of 11D10 can reduce the risk of HMFG-associated tumor occurrence, particularly in high risk individuals in the adjuvant setting. We also believe that administration of an anti-idiotype antibody for HMFG (such as 11D10) in conjunction with an anti-idiotype antibody for CEA (such as 3H1) can result in treatment of tumors which express both the HMFG and CEA antigens, for example in high risk individuals in adjuvant setting.

11D10 is a murine anti-idiotype (Id) antibody (Ab2) which induces a specific immune response against a distinct and specific epitope of human milk fat globule (HMFG), a tumor-associated antigen. The generation and characterization of 11D10 as well as the DNA sequences encoding the variable regions of 11D10 (light and heavy chains) has been described in commonly owned patent application no. 08/766,350 (attorney docket no. 30414/2000321). A hybridoma that produces 11D10 has been deposited with the American Type Culture Collection (ATCC), 12301 Parklawn Drive, Rockville, Md., U.S.A. 20852 on Jan. 17, 1996 under the provisions of the Budapest Treaty for the International Recognition of the Deposit of Microorganisms for the Purposes of Patent Procedure. It was accorded Accession No. HB12020.

In a previous Phase I clinical trial, 12 breast patients having advanced HMFG-associated disease (who had failed all previous therapy and still had high tumor burden) were administered 11D10. Chakraborty et al. (1997) Proc. Am. Ass. Cancer Research: 4139. Five of the 12 patients generated significant levels of specific anti-anti-Id (Ab3) antibodies that were capable of inhibiting binding of Ab2 to Ab1 or vice versa. This is especially significant, as many of these patients, either due to the nature of their previous treatment or their disease or both, are moderately to severely compromised, and often received 11D10 as a final option. Affinity purified Ab3 from 3 patients' sera bound specifically to the purified HMFG antigen and immunostained the breast cancer tissue sections. The isotype of the antibody (Ab3/Ab1′) was predominantly IgG. Peripheral blood lymphocytes (PBL) isolated from 3/12 immunized patients showed in vitro idiotype specific T cell proliferative responses. The results suggest that anti-ID 11D10 can induce both humoral and cellular immune responses in some advanced breast cancer patients who were heavily pretreated. Toxicity was minimal with only mild erythema and induration at the injection site. However, all of these patients displayed normal disease progression.

3H1 (which was previously disclosed in PCT Pubs. WO 96/20277 and WO 96/02019, both published on Jul. 4, 1996, which are herein incorporated in their entirety by reference) is a murine monoclonal anti-idiotype antibody which induces a specific immune response against a distinct and specific epitope of carcinoembryonic antigen (CEA), a tumor-associated antigen. This epitope is unique to CEA and is not present on other CEA-related lower molecular weight members of this family which are also found on normal tissues or hematopoietic cells including granulocytes. Koprowski et al. (1979) Somatic Cell Genet. 5:957; Mitchell (1980) Cancer Immunol. Immunother. 10:1. The antigenic determinant as defined by the monoclonal antibody 8019 (Ab1) against which 3H1 was raised is absent on normal adult tissues as evidenced by immunoperoxidase staining and hematopoietic analysis. The generation and characterization of 3H1 as well as the DNA sequences encoding the variable regions of 3H1 (light and heavy chains) has been described in commonly owned U.S. Pat. No. 5,977,315, which is incorporated herein in its entirety by reference. A hybridoma that produces 3H1 has been deposited with the American Type Culture Collection (ATCC), 12301 Parklawn Drive, Rockville, Md., U.S.A. 20852 on Dec. 15, 1995 under the provisions of the Budapest Treaty for the International Recognition of the Deposit of Microorganisms for the Purposes of Patent Procedure. It was accorded Accession No. HB12003.

Definitions

As used herein, the terms “11D10,” “11D10 antibody” and “11D10 monoclonal anti-idiotype antibody” are used interchangeably to refer to an anti-idiotype antibody (Ab2) which contains an epitope that at least partially resembles a distinct and specific epitope of human milk fat globule (HMFG) primarily expressed in human breast tumor cells. The generation and characterization of 11D10 is described in commonly owned patent application no. 08/766,350. See also Mukerjee et al. (1992) FASEB J. A2059 (Abs. 6505); Murkerjee et al. (1992) FASEB J. A1713 (Abs. 7792); Charaborty et al. (1994) Proc. Am. Assoc. for Cancer Res. 35:2963; Chakraborty et al. (1995) Cancer Res. 55:1525-1530; Bhattacharya-Chatterjee et al. (1994) Antigen and Antibody Mole. Eng. Breast Cancer Diagnosis and Treatment, (Ceriani, ed.) 139-148. Different biological functions are associated with 11D10, including, but not limited to, binding to Ab1 (MC-10) and/or Ab3 and an ability to induce an immune response (humoral and/or cellular) against HMFG in mice, rabbits, monkeys, and humans with advanced HMFG-associated disease, particularly HMFG-associated tumors, as well as humans with a history of HMFG-associated disease but no detectable disease. Also included in the definition of 11D10 are fragments produced by enzymatic cleavage and/or chemical treatment of intact antibody that comprise both the entire heavy and light chain variable regions of 11D10 and are capable of binding MC-10 (Ab1) in a standard immunoassay, such as Fab, F(ab′).sub.2, and F(ab′).

“HMFG” is an abbreviation for human milk fat globule. HMFG has several proteinaceous (and thus antigenic) components. As used herein, it refers to a semi-purified extract of an HMFG-expressing breast cancer cell line, as prepared by the method of Ceriani et al. ((1977) Proc. Natl. Acad. Sci. USA 74:582-586), along with antigenically related substances, including HMFG expressed on breast cancer cells and more highly purified preparations. Contained in HMFG is a high molecular weight mucin of known amino acid sequence, an epitope of which is recognized by the monoclonal antibody MC-10 used as Ab1 in raising 11D10. Accordingly, anti-HMFG immunological reactivity induced by immunizing an animal with 11D10 preferably binds a polypeptide epitope or an antigenic determinant related to that recognized by MC-10.

MC-10 was chosen for production of anti-Id because it defines a unique and specific epitope of a high molecular weight mucin of human milk fat globule (HMFG), primarily expressed at high density by human breast cancer and some other tumor cells but is not found on normal adult tissues by immunoperoxidase staining, or hematopoietic cells including granulocytes by flow cytometry analysis. MC-10 (also called BrE1) is quite restricted and specific in the sense that it reacts with a large molecular weight (MW 400,000) mucin present in only minute amounts in human mammary epithelial cells and increased by at least 10-fold on breast carcinoma cells. WO 8907268; EP 401247. The antibody is cytotoxic for breast cancer cells in in vitro studies. Ceriani et al. (1983); Peterson et al. (1990).

MC-10 has a very restricted histopathological distribution in normal tissues. MC-10 only binds some areas of the epithelial lining of the lung and scattered distal convoluted tubules of the kidney, with no apparent histopathological binding to normal breast and many other normal epithelia (colon, pancreas, stomach, thyroid, bladder, liver) and other normal tissues (adrenal, brain, lymph node, myocardium, ovary, spleen, testis). On the other hand, a high percentage of different human tumors, including breast, endometrium, lung, ovary, and pancreas bind mAb MC-10 intensely. The formalin fixed tumors studied for MC-10 binding (number positive/total number) include: breast carcinoma (CA) (144/182), colon CA (3/27), duodenum CA (0/1), endometrium CA (7/14), kidney CA (0/11), lung CA (41/47), ovary CA (20/26), pancreas CA (9/15), prostate CA (0/2), salivary gland CA (0/3), stomach CA (2/7), thyroid CA (0/7), hepatocholangio CA (8/33), islet cell CA (0/2), lymphoma (0/20), melanoma (0/23), meningioma (0/5), Merkel cell CA (4/9), mesothelioma (1/11), neuroblastoma (0/2), oncocytoma (1/1), paraganglioma (0/10), plleoadenoma (0/7). Among the sarcomas: unclassified (0/1), alveolar (0/1), angiosarcoma (0/1), clear cell (0/2), cystosarcoma (0/1), epithelioid (5/12), Ewing's (0/1), fibrosarcoma (0/1), leiomyoma (0/2), liposarcoma (0/1), malignant fibrohistiocytoma (0/2), synovial mesothelioma (0/7), spindle cell CA (5/16), undifferentiated (1/9); schwannoma (0/3), seminoma (0/4), teratoma (0/3), thymoma (0/8), transitional CA (5/10), undifferentiated CA (7/29), Warthin's tumor (0/1). Ceriani et al. (1990). We have also studied hematopoetic cells for the presence of MC-10 antigen by FACS analysis in our laboratory and found those cells, including granulocytes and platelets, negative for antigen. The positive control MCF-7 cells stained heavily with MC-10.

“An anti-idiotype antibody for HMFG,” as used herein, refers to an anti-idiotype antibody (Ab2) which contains an epitope that at least partially resembles a distinct and specific epitope of human milk fat globule (HMFG) primarily expressed in human breast tumor cells. Such an antibody is generally capable of eliciting an anti-HMFG immune response.

A “HMFG-associated tumor” is one that contains an HMFG antigen, especially expressed on the tumor cell surface, preferably that binds to MC-10 (Ab1). As noted above, this antigen is found on a wide variety of tumors particularly breast cancer (over 90% of breast cancer patients have tumors that react with MC-10). Thus, 11D10 has the potential to be used in a wide variety of cancers in which HMFG is detected. Methods of detecting HMFG are known in the art and examples are described herein. As used herein, “advanced” HMFG-associated tumors means that there is detectable metastasis, that is, detectable tumor masses at sites other than the primary site of the tumor. Masses are preferably detected by imaging techniques known in the art such as X-ray or CT scan.

As used herein, the terms “3H1”, “3H1 antibody” and “3H1 monoclonal anti-idiotype antibody” are used interchangeably to refer to immunoglobulin produced by the 3H1 hybridoma cell line deposited with the ATCC. Also included in the definition of 3H1 are fragments produced by enzymatic cleavage and/or chemical treatment of intact antibody that comprise both the entire heavy and light chain variable regions of 3H1 and are capable of binding 8019 (Ab1) in a standard immunoassay, such as Fab, F(ab′).sub.2, and F(ab′).

3H1 was obtained by using the 8019 antibody as immunogen for an anti-idiotype response. 8019 binds to a unique epitope of CEA that is not present on other members of the CEA family, with virtually no cross-reactivity with normal adult tissues or hematopoietic cells including granulocytes. Koprowski et al. (1979) Somatic Cell Genet. 5:957; Mitchell (1980) Cancer Immunol. Immunother. 10:1. The procedure for generation of monoclonal anti-idiotype hybridomas and selection of 3H1 are described in U.S. Pat. No. 5,977,315, incorporated herein by reference in its entirety.

We have found that 3H1 is effective in eliciting an immune response (humoral and/or cellular) in individuals with advanced CEA-associated tumors. While not wishing to be bound by a particular theory, one way that this may occur is that the 3H1 combining site may present a region that partly resembles an epitope in CEA, in the context of other epitopes which renders it more immunogenic. Thus, this antibody of this invention is useful for the treatment of CEA-associated tumors in these individuals. It is also useful for detection of Ab1 or Ab3.

“An anti-idiotype antibody for CEA,” as used herein, refers to an anti-idiotype antibody (Ab2) which contains an epitope that at least partially resembles a distinct and specific epitope of CEA which is an antigen expressed in many types of tumors (as described herein). Such an antibody is generally capable of eliciting an anti-CEA immune response.

As used herein, a “CEA-associated tumor” is one that expresses a CEA antigen, generally on the surface of tumor cells. As used herein, “advanced” CEA-associated tumors means that there is detectable metastasis, that is, detectable tumor masses at sites other than the primary site of the tumor. Masses are preferably detected by imaging techniques known in the art such as X-ray or CT scan.

As used herein, a “HMFG- and CEA-associated tumor” is a tumor that expresses both an HMFG antigen, generally expressed on the tumor cell surface, preferably that binds to MC-10, and a CEA antigen, generally expressed on the surface of tumor cells. The HMFG and CEA can be expressed by the same cell in a tumor (i.e., a single cell expressing both antigens), or by different cells in a tumor (i.e., cells expressing one antigen but not the other).

As used herein, “treatment” is an approach for obtaining beneficial or desired results. For purposes of this invention, beneficial or desired results include, but are not limited to, one or more of the following: alleviation of symptoms, diminishment of extent of disease, stabilized (i.e., not worsening) state of disease, preventing spread (i.e., metastasis) of disease, preventing occurrence or recurrence of disease, delay or slowing of disease progression, amelioration of the disease state, and remission (whether partial or total). Also encompassed by “treatment” is a reduction of pathological consequences of a HMFG- and CEA-associated tumor(s).

As used herein, “delaying” development of an HMFG- and CEA-associated tumor(s) means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of HMFG- and CEA-associated tumor(s) is a method that reduces probability of disease development in a given time frame and/or reduces extent of the disease in a given time frame, when compared to not using the method. Such comparisons are typically based on clinical studies, using a statistically significant number of subjects.

“Development” of HMFG- and CEA-associated tumor(s) means progression of the tumor(s). Tumor development can be detectable using standard clinical techniques as described herein. However, development also refers to disease progression that may be undetectable. For purposes of this invention, progression refers to the biological course of the disease state, in this case (i.e., HMFG- and CEA-associated tumors) cell division and/or metastasis of the HMFG- and CEA-associated tumor. “Development” includes occurrence, recurrence, and onset. As used herein “onset” or “occurrence” of HMFG- and CEA-associated disease includes initial onset and/or recurrence.

As used herein, “low tumor burden” means that an individual does not have advanced HMFG- and CEA-associated tumor(s). “Advanced” HMFG- and CEA-associated tumor(s) means that there is detectable metastasis, that is, detectable tumor masses at sites other than the primary site of the tumor. Tumor masses are generally detected by imaging techniques known in the art such as X-ray, CT scan, or MRI, as well as imaging and diagnostic techniques that detect tumor masses that would be detected by X-ray, CT scan, or MRI. As used herein, “advanced” disease does not include lymph node involvement. It is understood that “low tumor burden” also includes no detectable tumor using convention diagnostic techniques such as X ray, CT scan, or MRI. Preferably, an individual with low tumor burden has been assessed as having stage III, preferably stage II, even more preferably stage I disease. As described below, also preferable is disease that has been treated by surgery, radiation and/or chemotherapy and is no longer detectable by conventional diagnostic and/or imaging techniques. As another preferred example, individuals with “low tumor burden” also include those having surgical resection of the primary tumor in which no detectable disease or some disease remained due to, for example, inability to resect all detectable disease, or less extensive disease. Other examples of low tumor burden categories are provided below.

As used herein, a “high risk” individual is an individual who is at major risk of development of HMFG- and CEA-associated tumors. A “high risk” individual may or may not have detectable disease, and may or may not have displayed detectable disease prior to the treatment methods described herein. “High risk” denotes that an individual has one or more so-called risk factors, which are measurable parameters that correlate with development of HMFG- and CEA-associated tumors. An individual having one or more of these risk factors has a higher probability of developing HMFG- and CEA-associated tumors than an individual without these risk factor(s). These risk factors include, but are not limited to, age, sex, race, diet, history of previous disease, presence of precursor disease, genetic (i.e., hereditary) considerations, and environmental exposure. Examples (i.e., categories) of high-risk groups are discussed below.

Depending on the basis and context of assessment of high risk, the time frame within which probability of disease or tumor development, progression, and/or onset would more likely than not occur would vary. For instance, with breast cancer, high risk patients in the adjuvant setting, the risk of occurrence is typically measured within one to five years. For patients with non-small cell lung cancer in the adjuvant setting, the risk of occurrence is typically measured within one to two years. For patients who display precursor disease, the risk of occurrence can be measured in a longer time frame. For an individual who is considered high risk due to, for example, genetic or hereditary considerations, the risk of occurrence can be measured in an even longer time frame, including the expected lifetime of the individual.

An individual with “low risk” is one who is not considered “high risk”. “Adjuvant setting” refers to a setting in which an individual has had a history of HMFG- and CEA-associated disease, particularly HMFG- and CEA-associated tumors, and has been responsive to therapy. The prior therapy can have included, but is not limited to, surgical resection, radiotherapy, and chemotherapy. As a result of this prior therapy, these individuals have no clinically measurable tumor as detected by conventional diagnostic techniques such as X ray, CT scan, or MRI, or techniques that detect tumors detectable by X ray, CT scan, or MRI. However, because of their history of HMFG- and CEA-associated disease, these individuals are considered at risk of development of the disease. Treatment or administration in the “adjuvant setting” refers to a subsequent mode of treatment. The degree of risk (i.e., whether an individual in the adjuvant setting is considered “high risk” or “low risk”) depends upon several factors, most usually the extent of disease when first treated.

As used herein, “adjuvant setting” is distinguished from an “adjuvant”, which refers to a chemical or biological agent in a pharmaceutical preparation given in combination with an agent (such as an antibody, polynucleotide or polypeptide) to enhance its immunogenicity. Examples of adjuvants are described herein.

A “neo-adjuvant setting” refers to the period after diagnosis but before initiation of treatment modalities other than administration of 11D10 and 3H1. For example, if an individual is diagnosed as having a HMFG- and CEA-associated tumor, such as breast, for which surgery is indicated, administration of 11D10 and 3H1 in a neo-adjuvant setting means that administration of 11D10 and 3H1 commences before surgery.

An “effective amount” is an amount sufficient to effect beneficial or desired results, preferably within a clinical setting. An effective amount can be administered in one or more administrations. For purposes of this invention, an effective amount of 11D10 and 3H1 is an amount of 11D10 and 3H1 that is sufficient to ameliorate, stabilize, or delay the development or recurrence of the HMFG- and CEA-associated disease state, particularly HMFG- and CEA-associated tumors. A “beneficial or desired” result can also be elicitation of an immune response, whether humoral and/or cellular. Preferably, the immune response is the production of anti-CEA and/or anti-HMFG. Detection and measurement of these indicators of efficacy are discussed below.

An “individual” is a vertebrate, preferably mammal, more preferably human. Mammals include, but are not limited to, farm animals, sport animals, and pets.

As used herein, “in conjunction with” generally refers to in combination with (which can mean concurrent with, prior to, or after). Thus, as used herein, “the use of 11D10 in conjunction with 3H1”, or “the use of 3H1 in conjunction with 11D10”, or a “combination” of 11D10 and 3H1, refer to the administration of 11D10 and 3H1 to the same individual. Administration of 11D10 and 3H1 may be simultaneous, or one may be administered at a different time from the other. If 11D10 and 3H1 are administered simultaneously, they may be administered together in a single composition, or they may be administered in separate compositions. One may be administered more frequently than the other, in any permutation or combination. By way of example only, initial administration(s) to the individual may be in the form of simultaneous administration, but follow-up administrations may be of one or both of 11D10 and/or 3H1. 11D10 and/or 3H1 is administered “in conjunction with” other forms of therapy when an individual is given 11D10 and/or 3H1 concurrently with, prior to, or after other therapies.

Embodiments of the Invention

In one embodiment, the invention provides methods delaying development of an HMFG-associated tumor(s) in which an effective amount of 11D10 is administered to an individual having a low tumor burden. Examples of HMFG-associated tumors include, but are not limited to, breast carcinoma, ovarian carcinoma, non-small cell lung carcinoma, and pancreatic carcinoma. Methods of detecting HMFG-associated tumors are known in the art, including standard immunoassay and/or imaging techniques. As an example, HMFG-associated tumors can be detected by standard immunohistologic examination of affected tissue, using, for example, MC-10 as the primary antibody in an indirect immunofluorescence assay, FACS analysis, or immunoperoxidase staining assay.

In one embodiment, the invention encompasses administration of 11D10 to a high risk individual having a low tumor burden. As discussed above, a high risk individual displays one or more risk factors that correlate with HMFG-associated tumor development. High (i.e., increased) risk may be indicated, for example, on the basis of an individual's genotype (for example, presence of a gene(s) or mutations(s) that is associated with development of HMFG-associated tumors), increased expression of tumor-associated genes or decreased expression of tumor suppressor genes, presence of precursor disease (such as non-invasive masses), a family history of HMFG-associated cancer, a history of exposure to an environmental substance or form of radiation which is known or suspected of being carcinogenic or teratogenic (particularly suspected of causing HMFG-associated tumors), exposure to a potentially carcinogenic pathogen such as a retrovirus, or a history of other types of cancer or other types of abnormal or unregulated tissue growth. Also included as high risk are individuals suspected of having a HMFG positive tumor based on a positive test for anti-HMFG immunological reactivity. Such individual include those who may have had their primary tumor surgically removed and are at high risk because of the size of the primary tumor or the presence of positive lymph nodes.

Because all risk factors for developing HMFG-associated tumors are not known, and the interplay among these factors (in terms of overall risk) are not fully understood, it is clear to one skilled in the art that individuals suitable for administration of 11D10 for purposes of this invention can have features in common, and that individuals not falling clearly in the categories described above can nonetheless be considered suitable candidates for administration of 11D10. A skilled clinician can make an empirical determination whether an individual is suitable for 11D10 treatment. For example, an individual with a familial (i.e., genetic) history of breast cancer could be considered “high risk”, even though no previous disease in this individual has been observed. In this context, administration of 11D10 to such an individual could result in delay of occurrence of disease, to the extent that the individual does not develop the disease within his or her lifetime (or develops it later than would have been expected). Another example is an individual who is being treated using traditional modes of therapy, and who is showing responsiveness to the therapy (i.e., remission). Such an individual may be adjudged as “high risk”, even though the initial course of therapy is not yet completed, due to projection of progress by the administrator of the therapy, and can be a suitable candidate for receiving 11D10 before completion of the initial therapy. The discretion to determine whether treatment using 11D10 may be indicated is that of the person reponsible for the therapy.

It is also evident that administration of 11D10 may be indicated even if an individual is not adjudged to be high risk (i.e., is “low risk”) according to concurrent risk assessment criteria. For instance, an individual who has been successfully treated and is not considered high risk (due, for example, to the lack of detectable invasive disease at the time of diagnosis) may nonetheless be a candidate for receiving 11D10 as a precautionary measure, especially considering the lack of contraindications and lack of undesirable side effects so far observed from 11D10. Thus, the risk of disease progression may be lowered even further by administration of 11D10. As another example, an individual may believe that he or she is at risk of disease development, and may decide that receiving 11D10 would reduce this risk. Also suitable are individuals with supernormal levels of HMFG expression. Levels of HMFG expression can be determined by, for example, immunohistologic examination of affected tissue, using, for example, MC-10 as the primary antibody in an indirect immunofluoresence assay.

In another embodiment of the present invention, 11D10 is administered to a high risk individual in the adjuvant setting. Factors typical as indicating individuals of high risk in the adjuvant setting are invasion by the tumor into neighboring tissues (i.e., extensive disease), and/or lymph node involvement. Examples of high risk individuals in the adjuvant setting include, but are not limited to, (a) patients with Stage II or Stage IIIA non-small lung cancer (NSCLC) who have had their tumor resected but have positive regional lymph nodes (these patients have a 60-80% relapse rate in the first 2 years); and (b) patients with breast cancer who have positive lymph nodes in preferably at least 5, more preferably at least 10 positive lymph nodes (70-80% relapse rate in the first 2 years for those with at least 10 positive lymph nodes). Another example of a high risk individual in the adjuvant setting is an individual having ovarian cancer which is an HMFG-associated tumor and has detectable disease post-surgery. This post-surgery detectable disease, generally due to unresectable disease, is generally visually detected (for example, when a patient is in surgery), although its presence can be based on other methods of detection, such as CT scan.

In another embodiment, 11D10 is administered in a neo-adjuvant setting. It is understood that, for purposes of this invention, an individual in a neo-adjuvant setting has a low tumor burden. Preferably, when administered in the neo-adjuvant setting, an individual has low tumor mass.

Another example of an individual suitable for 11D10 therapy as described in this invention is an individual with low tumor burden. Thus, the present invention encompasses methods of treating HMFG-associated tumors in an individual having a low tumor burden comprising administering an effective amount of HMFG. As defined above, a “low” tumor burden means that the disease is not considered advanced. For example, a low tumor burden can be disease in partial or complete remission as adjudged by a clinical practitioner. “Low” tumor burden can also arise by a reduction of tumor burden of advanced disease such that the extent of disease is no longer considered advanced. Other examples of low tumor burden include disease contained to limited lymph node involvement. An individual with a low tumor burden can be further classified as “high risk” or “low risk,” depending on the individual's history of disease and treatment. As one skilled in the art would readily appreciate, an individual with low tumor burden could be treated in the non-adjuvant, neo-adjuvant, and/or adjuvant setting(s).

The invention also includes methods of treatment using 11D10 for individuals having residual disease, particularly minimal residual disease. “Residual” disease is any 11D10 associated disease, particularly HMFG associated tumor(s) remaining after therapy but which is undetectable by conventional diagnostic techniques such as X ray, CT scan, or MRI, or techniques that detect tumors detectable by X ray, CT scan or MRI. Thus, “residual disease” refers to the likely presence of disease that can develop into detectable disease, and refers to a prognosis and/or assumption made in an adjuvant setting. Depending on the type of HMFG-associated tumor and, for example, the extent of disease upon diagnosis, an individual can be adjudged to have residual disease, even though no detectable disease is present. For example, an individual with resectable NSCLC has residual disease after surgery (i.e., resection), even if an apparent complete remission has occurred. Similarly, an individual with breast cancer can have micrometastatic residual disease after chemotherapy. Alternatively, an individual who is currently undergoing therapy for an HMFG-associated tumor also has “residual” disease. It is understood that, as used herein, “residual” disease does not include advanced disease. “Residual” disease and “minimal residual” disease as used herein are both undetectable using conventional diagnostic techniques such as X ray, CT scan, or MRI, or techniques that detect tumors detectable by X ray, CT scan or MRI, but refer to varying extent or degrees of the disease.

The invention also encompasses methods of reducing risk of occurrence of HMFG-associated disease, particularly HMFG-associated tumors. In these methods, an effective amount of 11D10 is administered to an individual at risk for developing HMFG-associated disease. “Reducing risk of occurrence” means that the risk of occurrence and/or reoccurrence of HMFG-associated disease is lower in individuals receiving 11D10 than those individuals (having the same risk of occurrence) who do not. An individual “at risk” for developing HMFG-associated disease can be high risk or low risk, depending on the clinical and genetic history and status of the individual.

In another embodiment, the invention provides methods of treating an HMFG-associated tumor, particularly breast cancer, which include administration of certain chemotherapeutic agents and 11D10. We believe that certain chemotherapeutic agents may act synergistically with 11D10 to enhance the immune response. Appropriate chemotherapeutic agents may be determined based on data indicating that the chemotherapeutic agent(s) may stimulate the immune response, or not diminish the immune response. Methods of measuring the immune response are known in the art and are described herein. Administration of these chemotherapeutic agents generally follow accepted clinical protocols.

For all of the above-described embodiments of the present invention, 11D10 can be prepared, administered, and monitored as described in the following sections.

In one embodiment, the invention provides methods of delaying development of an HMFG- and CEA-associated tumor(s) in which an effective amount of 11D10 and 3H1 is administered to an individual, preferably an individual having a low tumor burden. Examples of HMFG- and CEA-associated tumors include, but are not limited to, breast cancer, ovarian cancer, small cell lung carcinoma, non-small cell lung carcinoma, and colorectal cancer. Methods of detecting HMFG- and CEA-associated tumors are known in the art, including standard immunoassay and/or imaging techniques. As an example, HMFG- and CEA-associated tumors can be detected by standard immunohistologic examination of affected tissue, using, for example, BrE1 (Mc10) as the primary antibody for HMFG, and antibody 8019 as the primary antibody for CEA, in an indirect immunofluorescence assay, FACS analysis, or immunoperoxidase staining assay.

In one embodiment, the invention encompasses administration of a combination of 11D10 and 3H1 to a high risk individual having a low tumor burden. As discussed above, a high risk individual displays one or more risk factors that correlate with HMFG- and CEA-associated tumor development. High (i.e., increased) risk may be indicated, for example, on the basis of an individual's genotype (for example, presence of a gene(s) or mutations(s) that is associated with development of HMFG- and CEA-associated tumors), increased expression of tumor-associated genes or decreased expression of tumor suppressor genes, presence of precursor disease (such as non-invasive masses), a family history of HMFG- and CEA-associated cancer, a history of exposure to an environmental substance or form of radiation which is known or suspected of being carcinogenic or teratogenic (particularly suspected of causing HMFG- and CEA-associated tumors), exposure to a potentially carcinogenic pathogen such as a retrovirus, or a history of other types of cancer or other types of abnormal or unregulated tissue growth. Also included as high risk are individuals suspected of having a HMFG- and CEA positive tumor based on a positive test for anti-HMFG and/or anti-CEA immunological reactivity. Such individual include those who may have had their primary tumor surgically removed and are at high risk because of the size of the primary tumor or the presence of positive lymph nodes.

Because all risk factors for developing HMFG- and CEA-associated tumors are not known, and the interplay among these factors (in terms of overall risk) are not fully understood, it is clear to one skilled in the art that individuals suitable for administration of a combination 11D10 and 3H1 for purposes of this invention can have features in common, and that individuals not falling clearly in the categories described above can nonetheless be considered suitable candidates for administration of a combination of 11D10 and 3H1. A skilled clinician can make an empirical determination whether an individual is suitable for combination 11D10 and 3H1 treatment. For example, an individual with a familial (i.e., genetic) history of breast cancer could be considered “high risk”, even though no previous disease in this individual has been observed. In this context, administration of a combination of 11D10 and 3H1 to such an individual could result in delay of occurrence of disease, to the extent that the individual does not develop the disease within his or her lifetime (or develops it later than would have been expected). Another example is an individual who is being treated using traditional modes of therapy, and who is showing responsiveness to the therapy (i.e., remission). Such an individual may be adjudged as “high risk”, even though the initial course of therapy is not yet completed, due to projection of progress by the administrator of the therapy, and can be a suitable candidate for receiving a combination of 11D10 and 3H1 before completion of the initial therapy. The discretion to determine whether treatment using a combination of 11D10 and 3H1 may be indicated is that of the person reponsible for the therapy.

It is also evident that administration of a combination of 11D10 and 3H1 may be indicated even if an individual is not adjudged to be high risk (i.e., is “low risk”) according to concurrent risk assessment criteria. For instance, an individual who has been successfully treated and is not considered high risk (due, for example, to the lack of detectable invasive disease at the time of diagnosis) may nonetheless be a candidate for receiving a combination of 11D10 and 3H1 as a precautionary measure, especially considering the lack of contraindications and lack of undesirable side effects so far observed from a combination of 11D10 and 3H1. Thus, the risk of disease progression may be lowered even further by administration of a combination of 11D10 and 3H1. As another example, an individual may believe that he or she is at risk of disease development, and may decide that receiving a combination of 3H1 and 11D10 would reduce this risk. Also suitable are individuals with supernormal levels of HMFG and/or CEA expression. Levels of HMFG expression can be determined by, for example, immunohistologic examination of affected tissue, using, for example, BrE1 (MC-10) as the primary antibody in an indirect immunofluoresence assay. Levels of CEA expression can be determined by, for example, immunohistologic examination of affected tissue, using, for example, antibody 8019.

In another embodiment of the present invention, a combination of 11D10 and 3H1 is administered to a high risk individual in the adjuvant setting. Factors typical as indicating individuals of high risk in the adjuvant setting are invasion by the tumor into neighboring tissues (i.e., extensive disease), and/or lymph node involvement.

Examples of high risk individuals in the adjuvant setting include, but are not limited to, (a) patients with Stage II or Stage IIIA non-small lung cancer (NSCLC) who have had their tumor resected but have positive regional lymph nodes (these patients have a 60-80% relapse rate in the first 2 years); and (b) patients with breast cancer who have positive lymph nodes in preferably at least 5, more preferably at least 10 positive lymph nodes (70-80% relapse rate in the first 2 years for those with at least 10 positive lymph nodes). Another example of a high risk individual in the adjuvant setting is an individual having ovarian cancer which is a HMFG- and CEA-associated tumor and has detectable disease post-surgery. This post-surgery detectable disease, generally due to unresectable disease, is generally visually detected (for example, when a patient is in surgery), although its presence can be based on other methods of detection, such as CT scan. Yet another example of a high risk individual in the adjuvant setting is an individual having colon cancer with at least 4 positive lymph nodes (70-80% relapse rate in the first 2 years). Still another example of a high risk individual in the adjuvant setting is an individual having a small cell lung carcinoma that has been resected.

In another embodiment, a combination of 11D10 and 3H1 is administered in a neo-adjuvant setting. It is understood that, for purposes of this invention, an individual in a neo-adjuvant setting has a low tumor burden. Preferably, when administered in the neo-adjuvant setting, an individual has low tumor mass.

Another example of an individual suitable for combination 11D10 and 3H1 therapy as described in this invention is an individual with low tumor burden. Thus, the present invention encompasses methods of treating HMFG- and CEA-associated tumors in an individual having a low tumor burden comprising administering an effective amount of HMFG and CEA. As defined above, a “low” tumor burden means that the disease is not considered advanced. For example, a low tumor burden can be disease in partial or complete remission as adjudged by a clinical practitioner. “Low” tumor burden can also arise by a reduction of tumor burden of advanced disease such that the extent of disease is no longer considered advanced. Other examples of low tumor burden include disease contained to limited lymph node involvement. An individual with a low tumor burden can be further classified as “high risk” or “low risk,” depending on the individual's history of disease and treatment. As one skilled in the art would readily appreciate, an individual with low tumor burden could be treated in the non-adjuvant, neo-adjuvant, and/or adjuvant setting(s).

The invention also includes methods of treatment using a combination of 11D10 and 3H1 for individuals, preferably those individuals having residual disease, particularly minimal residual disease. “Residual” disease is any 11D10 and 3H1-associated disease, particularly HMFG- and CEA-associated tumor(s) remaining after therapy but which is undetectable by conventional diagnostic techniques such as X ray, CT scan, or MRI, or techniques that detect tumors detectable by X ray, CT scan or MRI. Thus, “residual disease” refers to the likely presence of disease that can develop into detectable disease, and refers to a prognosis and/or assumption made in an adjuvant setting. Depending on the type of HMFG- and CEA-associated tumor and, for example, the extent of disease upon diagnosis, an individual can be adjudged to have residual disease, even though no detectable disease is present. For example, an individual with resectable NSCLC has residual disease after surgery (i.e., resection), even if an apparent complete remission has occurred. Similarly, an individual with breast cancer can have micrometastatic residual disease after chemotherapy. Alternatively, an individual who is currently undergoing therapy for an HMFG- and CEA-associated tumor also has “residual” disease. It is understood that, as used herein, “residual” disease does not include advanced disease. “Residual” disease and “minimal residual” disease as used herein are both undetectable using conventional diagnostic techniques such as X ray, CT scan, or MRI, or techniques that detect tumors detectable by X ray, CT scan or MRI, but refer to varying extent or degrees of the disease.

The invention also encompasses methods of reducing risk of occurrence of HMFG- and CEA-associated disease, particularly HMFG- and CEA-associated tumors. In these methods, an effective amount of a combination of 11D10 and 3H1 is administered to an individual at risk for developing HMFG- and CEA-associated disease. “Reducing risk of occurrence” means that the risk of occurrence and/or reoccurrence of HMFG- and CEA-associated disease is lower in individuals receiving a combination of 11D10 and 3H1 than those individuals (having the same risk of occurrence) who do not. An individual “at risk” for developing HMFG- and CEA-associated disease can be high risk or low risk, depending on the clinical and genetic history and status of the individual.

In another embodiment, the invention provides methods of treating an HMFG- and CEA-associated tumor, particularly breast cancer, which include administration of certain chemotherapeutic agents and a combination of 11D10 and 3H1. We believe that certain chemotherapeutic agents may act synergistically with 11D10 and 3H1 to enhance the immune response. Appropriate chemotherapeutic agents may be determined based on data indicating that the chemotherapeutic agent(s) may stimulate the immune response, or not diminish the immune response. Methods of measuring the immune response are known in the art and are described herein. Administration of these chemotherapeutic agents generally follow accepted clinical protocols.

For all of the above-described embodiments of the present invention, a combination of 11D10 and 3H1 can be prepared, administered, and monitored as described in the following sections.

Some embodiments of this invention entail administration of an effective amount of 11D10 and 3H1. The administration may be simultaneous, either in the form of a single composition comprising both 11D11 and 3H1, or in the form of administration of separate compositions for each. Alternatively, the administration of 11D10 and 3H1 may not be simultaneous, or may be simultaneous for only part of the course of administration. One of the two anti-idiotype antibodies may be administered more frequently and/or in greater quantities than the other. The invention encompasses all permutations and combinations of separate and simultaneous administration of 11D10 and 3H1.

Preparation of Anti-idiotype Antibody 11D10

11D10 can be obtained several ways. For example, 11D10 can be produced from the hybridoma ATCC No. HB 12020 described herein, or progeny thereof. As used herein, “progeny” of a hybridoma are descendants of a hybridoma, which may or may not be completely identical to the original (parent) cell due to mutation or other adaptation, but that produce a monoclonal antibody that maintains the ability to escape immune tolerance, i.e., to cause an immune reaction against HMFG. Methods of antibody isolation are well known in the art. See, for example, Harlow and Lane (1988) Antibodies: A Laboratory Manual, Cold Spring Harbor Laboratory, New York, and Sambrook et al. (1989) Molecular Cloning: A Laboratory Manual, Cold Spring Harbor Laboratory. The antibody can be obtained from the hybridoma via tissue culture or from mouse ascites. These techniques are known in the art. For example, the cells can be cultured in a suitable medium, and spent medium can be used as an antibody source. Optionally, matrix-coated channels or beads and cell co-cultures may be included to enhance growth of antibody-producing cells. For the production of large amounts of antibody, it is generally more convenient to obtain an ascites fluid. Such methods are known in the art, and generally comprise injecting hybridoma cells into an immunologically naive histocompatible or immunotolerant mammal, especially a mouse. The mammal is optionally primed for ascites production by prior administration of a suitable composition; for example, Pristane. Preferably, 11D10 is purified from BALB/c ascites using recombinant protein G-agarose chromatography followed by Protein-A-CL-sepharose 4B chromatography.

Alternatively, 11D10 can be chemically synthesized using techniques known in the art, for example, using a commercially available automated peptide synthesizer such as those manufactured by Applied Biosystems, Inc. (Foster City, Calif.).

11D10 can also be obtained by employing routine recombinant methods such as described in Sambrook et al. (1989). For instance, a polynucleotide encoding either the 11D10 heavy or light chain can be cloned into a suitable expression vector (which contains control sequences for transcription, such as a promoter). The expression vector is in turn introduced into a host cell. The host cell is grown under suitable conditions such that the polynucleotide is transcribed and translated into a protein. Heavy and light chains of 11D10 may be produced separately, and then combined by disulfide bond rearrangement. Alternatively, vectors with separate polynucleotides encoding each chain of 11D10, or a vector with a single polynucleotide encoding both chains as separate transcripts, may be transfected into a single host cell which may then produce and assemble the entire molecule. Preferably, the host cell is a higher eukaryotic cell that can provide the normal carbohydrate complement of the molecule. The 11D10 thus produced in the host cell can be purified using standard techniques in the art.

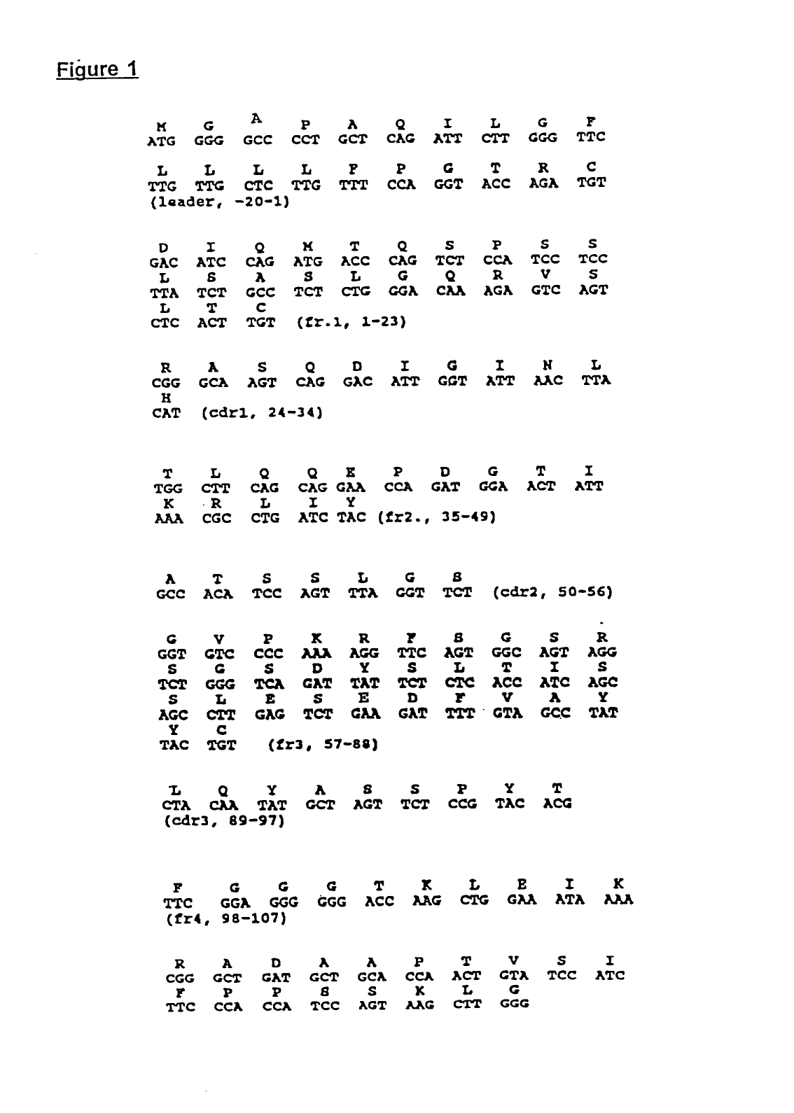

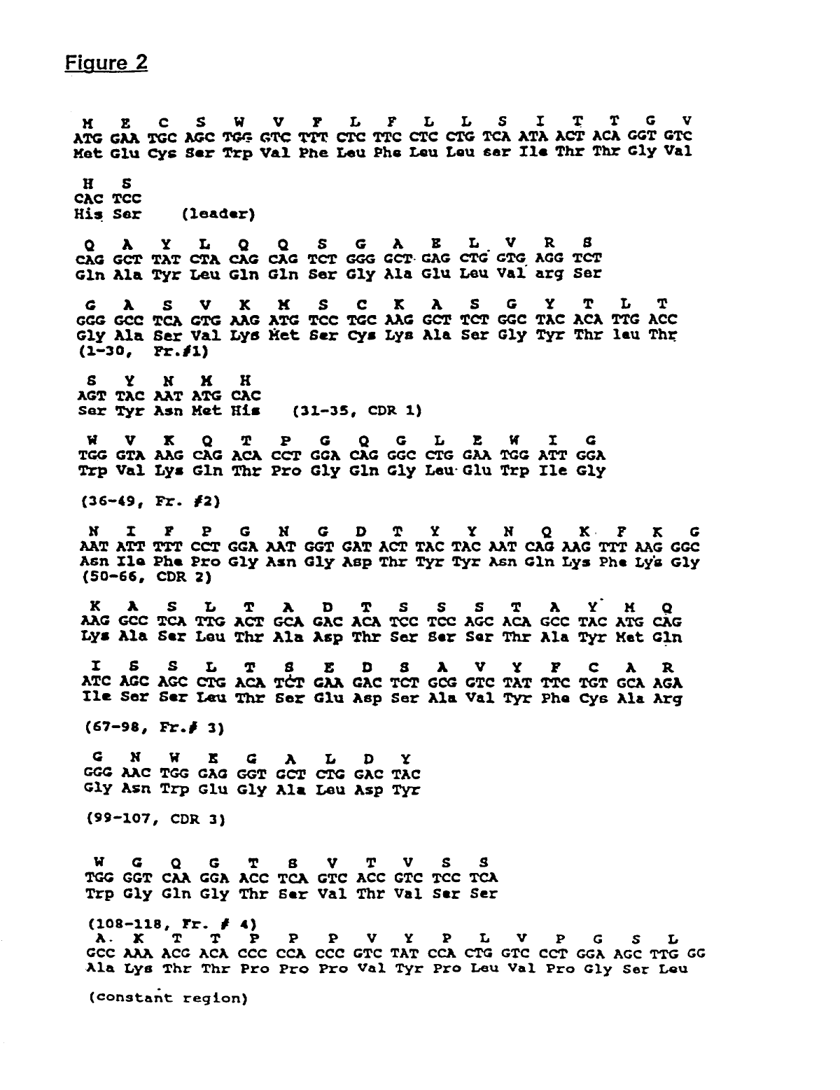

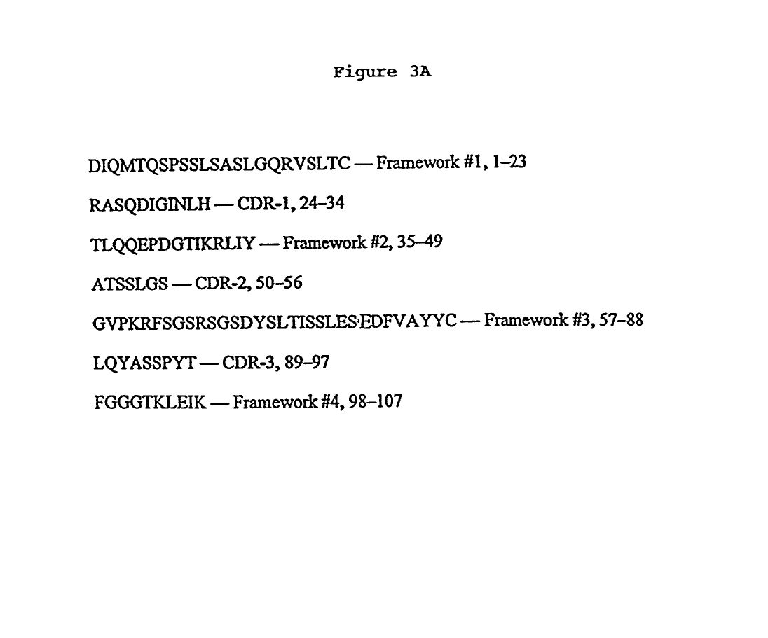

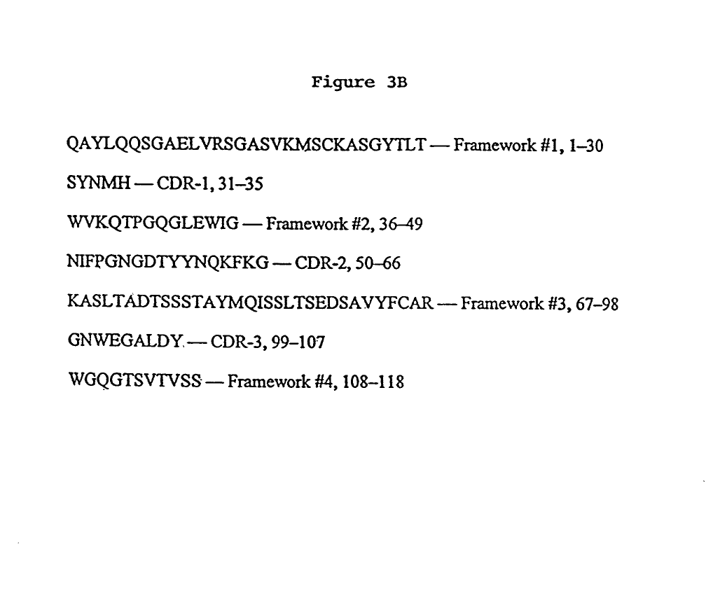

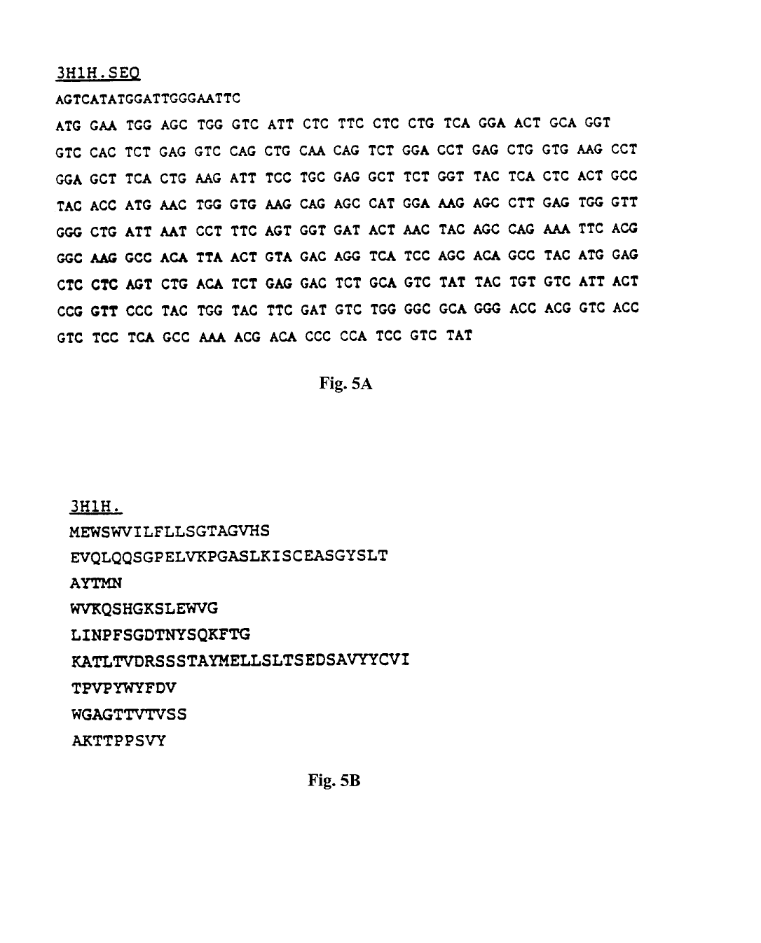

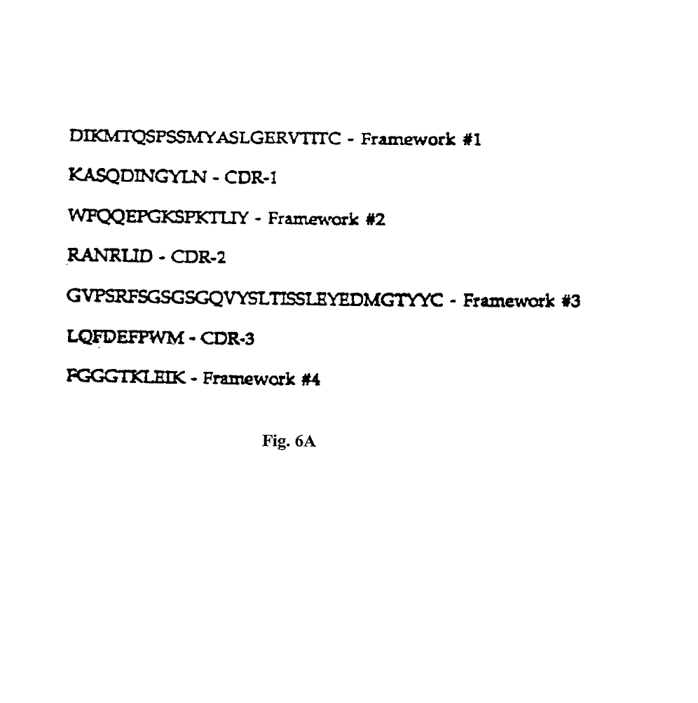

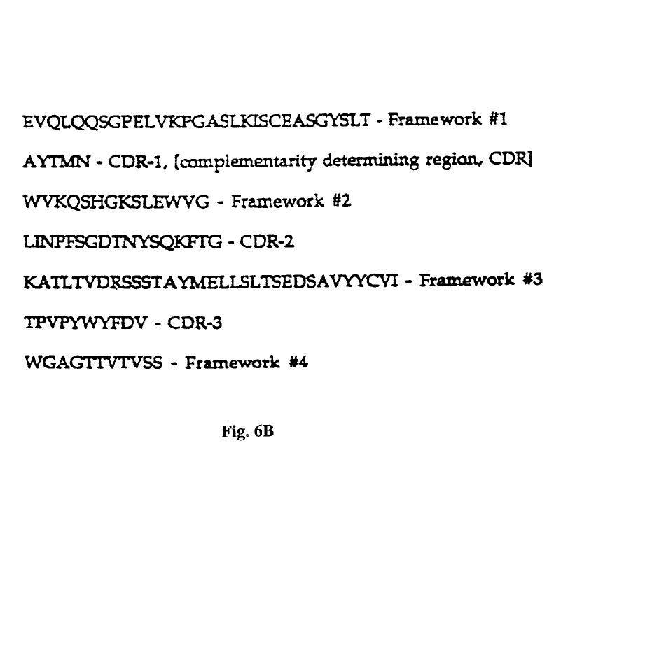

A polynucleotide encoding 11D10 for use in the production of 11D10 by any of these methods can in turn be obtained from the hybridoma producing 11D10, or be produced synthetically or recombinantly from the DNA sequences described in commonly owned patent application nos. 08/766,350 (attorney docket no. 30414/2000321) using standard techniques in the art. FIG. 1 depicts the cDNA sequence of the light chain variable region of 11D10 (SEQ ID NO:1); FIG. 2 depicts the cDNA sequence of the heavy chain variable region of 11D10 (SEQ ID NO:3). The full sequences of the 11D10 light and heavy chain constant regions have not been determined, but are expected to be identical or substantially identical to those of other mouse immunoglobulin molecules. For the mouse kappa light chain constant region, four genetic allotypes encoding two protein allotypes have been published by Solin et al. (1993) Immunogenetics 37:401-407, which is hereby incorporated herein by reference. FIG. 1 of Solin et al. depicts mouse and rat immunoglobulin kappa chain gene sequences, comparing the sequences within the kappa chain constant region for different strains and highlighting allotypic differences. Included are kappa chain constant region sequences for BALB/c, PL, SJL, and M. spretus. Other naturally occurring allotypes are possible. The mouse γ₁ heavy chain constant region DNA sequence from newborn mice has been published by Honjo et al. (1979) Cell 18:559-568, which is hereby incorporated herein by reference. FIG. 5 of Honjo et al. shows the germ-line DNA sequence, along with the encoded protein sequence. Shown in the line above is another protein sequence obtained from the mouse myeloma MOPC 21. Other naturally occurring allotypes are possible.

Polynucleotides encoding 11D10 can also be derived from the amino acid sequence of 11D10, the variable regions of which are provided in FIG. 1 (light chain; SEQ ID NO:2) and FIG. 2 (heavy chain; SEQ ID NO:4). Given the amino acid sequence of 11D10, one of skill in the art can design polynucleotides encoding The 11D10 antibody isolated from hybridoma ATCC No. HB 12020 is of the IgG1 mouse subclass, and may be isolated by any technique suitable for immunoglobulins of this isotype. Purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-immunoglobulin. 11D10 may also be purified on affinity columns comprising the MC-10 (BrE1) paratope; for example, in the form of a purified Ab1 or Ab3.

As would be evident to one skilled in the art, other anti-idiotype antibodies for HMFG can be generated using methods described herein for generating 11D10.

In some embodiments, methods of the invention utilize an anti-idiotype antibody for HMFG having a light chain variable region amino acid sequence identical to that depicted in FIG. 1B (SEQ ID NO:2) and a heavy chain variable region amino acid sequence identical to that depicted in FIG. 2B (SEQ ID NO:4). The invention also encompasses an anti-idiotype antibody for HMFG having a light chain variable region encoded by a polynucleotide sequence identical to that depicted in FIG. 1A (SEQ ID NO:1) and a heavy chain variable region encoded by a polynucleotide sequence identical to that depicted in FIG. 2A (SEQ ID NO:3). These antibodies can be prepared using methods described above for preparing 11D10.

An “identical” polynucleotide or amino acid sequence means that, when the sequences are aligned, there is an exact match between bases (polynucleotide) or amino acids.

In other embodiments, methods of the invention utilize an anti-idiotype antibody for HMFG having a light chain variable region encoded by a polynucleotide encoding an amino acid sequence identical to that depicted in FIG. 1B (SEQ ID NO:2) and a heavy chain variable region encoded by a polynucleotide encoding an amino acid sequence identical to that depicted in FIG. 2B (SEQ ID NO:4). It is well within the skill of the art, given an amino acid sequence, to deduce a polynucleotide encoding the amino acid sequence.

In some embodiments, methods of the invention utilize a polypeptide having immunological activity of 11D10, wherein the polypeptide comprises an immunoglobulin variable region containing at least one light chain complementarity determining region (CDR) of 11D10, and/or an immunoglobulin variable region containing at least one heavy chain CDR of 11D10, wherein the immunological activity of the polypeptide is an ability to stimulate a specific immune response against HMFG. Methods of preparing these polypeptides can be prepared using methods known in the art, including those described above for preparing 11D10.

Preparation of Anti-idiotype Antibody 3H1

The 3H1 antibody can be obtained in several ways. 3H1 can be produced from the hybridoma ATCC No. HB112003 described herein, or progeny thereof. As used herein, “progeny” of a hybridoma are descendants of a hybridoma, which may or may not be completely identical to the original (parent) cell due to mutation or other adaptation, but that produce a monoclonal antibody that maintains the ability to escape immune tolerance, i.e., to cause an immune reaction against CEA. Methods of antibody isolation are well known in the art. See, for example, Harlow and Lane (1988) Antibodies: A Laboratory Manual, Cold Spring Harbor Laboratory, New York, and Sambrook et al. (1989) Molecular Cloning: A Laboratory Manual, Cold Spring Harbor Laboratory. The antibody can be obtained from the hybridoma via tissue culture or from mouse ascites. These techniques are known the art. For example, the cells can be cultured in a suitable medium, and spent medium can be used as an antibody source. Optionally, matrix-coated channels or beads and cell co-cultures may be included to enhance growth of antibody-producing cells. For the production of large amounts of antibody, it is generally more convenient to obtain an ascites fluid. Such methods are known in the art, and generally comprise injecting hybridoma cells into an immunologically naive histocompatible or immunotolerant mammal, especially a mouse. The mammal is optionally primed for ascites production by prior administration of a suitable composition, for example, Pristane. Preferably, 3H1 is purified from BALB/c ascites using recombinant protein G-agarose chromatography followed by Protein-A-CL-sepharose 4B chromatography.

Alternatively, 3H1 can be chemically synthesized using techniques known in the art, for example, using a commercially available automated peptide synthesizer such as those manufactured by Applied Biosystems, Inc. (Foster City, Calif.). 3H1 can also be obtained by employing routine recombinant methods such as described in Sambrook et al. (1989). For instance, a polynucleotide encoding either the 3H1 heavy or light chain can be cloned into a suitable expression vector (which contains control sequences for transcription, such as a promoter). The expression vector is in turn introduced into a host cell. The host cell is grown under suitable conditions such that the polynucleotide is transcribed and translated into a protein. Heavy and light chains of 3H1 may be produced separately, and then combined by disulfide bond rearrangement. Alternatively, vectors with separate polynucleotides encoding each chain of 3H1, or a vector with a single polynucleotide encoding both chains as separate transcripts, may be transfected into a single host cell which may then produce and assemble the entire molecule. Preferably, the host cell is a higher eucaryotic cell that can provide the normal carbohydrate complement of the molecule. The 3H1 thus produced in the host cell can be purified using standard techniques in the art. A polynucleotide encoding 3H1 for use in the production of 3H1 by any of these methods can in turn be obtained from the hybridoma producing 3H1, or be produced synthetically or recombinantly from the DNA sequence provided herein.

The 3H1 antibody is of the IgG1 mouse subclass, and may be isolated by any technique suitable for immunoglobulins of this isotype. Purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-immunoglobulin. 3H1 may also be purified on affinity columns comprising the 8019 paratope; for example, in the form of a purified Ab1 or Ab3.

As would be evident to one skilled in the art, other anti-idiotype antibodies for CEA can be generated using methods described herein for generating 3H1.

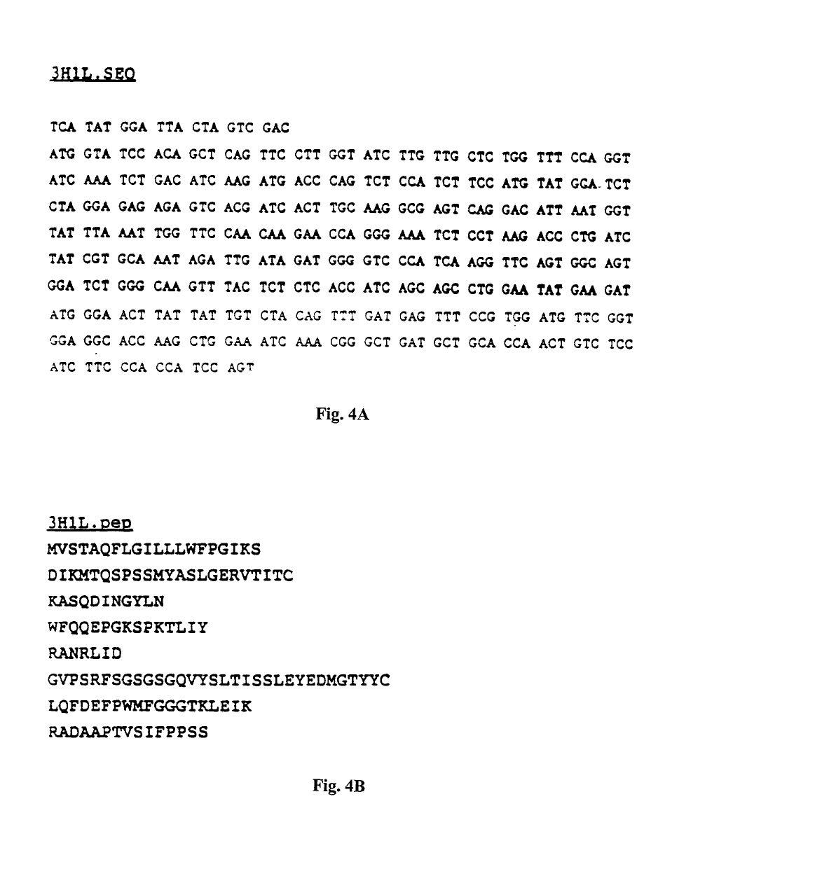

In some embodiments, methods of the invention utilize an anti-idiotype antibody for CEA having a light chain variable region amino acid sequence identical to that depicted in FIG. 4B (SEQ ID NO:6) and a heavy chain variable region amino acid sequence identical to that depicted in FIG. 5B (SEQ ID NO:8). The invention also encompasses an anti-idiotype antibody for CEA having a light chain variable region encoded by a polynucleotide sequence identical to that depicted in FIG. 4A (SEQ ID NO:5) and a heavy chain variable region encoded by a polynucleotide sequence identical to that depicted in FIG. 5A (SEQ ID NO:7). These antibodies can be prepared using methods described above for preparing 3H1.

An “identical” polynucleotide or amino acid sequence means that, when the sequences are aligned, there is an exact match between bases (polynucleotide) or amino acids.

In other embodiments, methods of the invention utilize an anti-idiotype antibody for CEA having a light chain variable region encoded by a polynucleotide encoding an amino acid sequence identical to that depicted in FIG. 1B (SEQ ID NO:2) and a heavy chain variable region encoded by a polynucleotide encoding an amino acid sequence identical to that depicted in FIG. 2B (SEQ ID NO:4). It is well within the skill of the art, given an amino acid sequence, to deduce a polynucleotide encoding the amino acid sequence.

In some embodiments, methods of the invention utilize a polypeptide having immunological activity of 3H1, wherein the polypeptide comprises an immunoglobulin variable region containing at least one light chain complementarity determining region (CDR) of 3H1, and/or an immunoglobulin variable region containing at least one heavy chain CDR of 3H1, wherein the immunological activity of the polypeptide is an ability to stimulate a specific immune response against CEA. Methods of preparing these polypeptides can be prepared using methods known in the art, including those described above for preparing 3H1.

Administration of 11D10 in conjunction with 3H1

If 11D10 and 3H1 are to be administered to an individual, the 11D10 and the 3H1, as separate preparations or combined in one preparation, are preferably at least 80% pure, more preferably at least 90% pure, even more preferably at least 95% pure, even more preferably at least 98% pure, as well as free of pyrogens and other contaminants. In this context, the percent purity is calculated as a weight percent of the total protein content of the preparation. Preparation of 11D10 and 3H1 for immunization is described in Example 1.

Preferably, 11D10 and 3H1 are administered with a pharmaceutically acceptable excipient. A pharmaceutically acceptable excipient is a relatively inert substance that facilitates administration of a pharmacologically effective substance. For example, an excipient can give form or consistency to the vaccine composition, or act as a diluent. Suitable excipients include but are not limited to stabilizing agents, wetting and emulsifying agents, salts for varying osmolarity, encapsulating agents, buffers, and skin penetration enhancers. Examples of pharmaceutically acceptable excipients are described in Remington's Pharmaceutical Sciences (Alfonso R. Gennaro, ed., 18th edition, 1990).

Preferably, the 11D10 and 3H1 are used with an adjuvant which enhances presentation of 11D10 and 3H1 or otherwise enhances the immune response against 11D10 and 3H1. Suitable adjuvants include aluminum hydroxide, alum, QS-21 (U.S. Pat. No. 5,057,540), DHEA (U.S. Pat. Nos. 5,407,684 and 5,077,284) and its derivatives (including salts) and precursors (e.g., DHEA-S), beta-2 microglobulin (WO 91/16924), muramyl dipeptides, muramyl tripeptides (U.S. Pat. No. 5,171,568), monophosphoryl lipid A (U.S. Pat. No. 4,436,728; WO 92/16231) and its derivatives (e.g., DETOX™), and BCG (U.S. Pat. No. 4,726,947). Other suitable adjuvants include, but are not limited to, aluminum salts, squalene mixtures (SAF-1), muramyl peptide, saponin derivatives, mycobacterium wall preparations, mycolic acid derivatives, nonionic block copolymer surfactants, Quil A, cholera toxin B subunit, polyphosphazene and derivatives, and immunostimulating complexes (ISCOMs) such as those described by Takahashi et al. (1990) Nature 344:873-875. For veterinary use and for production of antibodies in animals, mitogenic components of Freund's adjuvant can be used. The choice of an adjuvant will depend in part on the stability of the vaccine in the presence of the adjuvant, the route of administration, and the regulatory acceptability of the adjuvant, particularly when intended for human use. For instance, alum is approved by the United States Food and Drug Administration (FDA) for use as an adjuvant in humans. Preferably, alum-precipitated 11D10 and 3H1 are used. Preparation of aluminum hydroxide precipitated 11D10 and 3H1 is described in Example 1. Preferably, QS-21 (i.e., STIMULON™ QS-21, Acquila Biotech, Worcester, Mass.) or DETOX™ PC (Ribi Immunochem, Hamilton, Mont.) is used.

STIMULON™ QS-21, available from Acquila Biotech (formerly Cambridge Biotech Corp.), Worcester, Mass., is a component of the extract from the tree Quillaja saponaria Molina. The QS-21 molecule (C₉₂H₁₄₈O₄₆, M.W. 1990) consists of a triterpene glycoside with the general structure of a quillaic acid 3,28-O-his glycoside. It consists of two structural isomers designed V1 and V2 at a typical ratio of V1:V2 of approximately 2:1. Preferably, 100 μg of STIMULON™ QS-21 is used per administration of 11D10 and/or 3H1.

DETOX™PC, available commercially from Ribi Immunobiochem (Hamilton, Mont.) is a mixture of cell wall skeleton (CWS) from Mycobacterium phlei and Monophosphoryl Lipid A (MPL®) from Salmonella minnesota Re595 prepared as stable oil-in-water emulsion with squalane, Tween-80 saline, egg phsophatidylcholine and α-tocopherol. The ration of CWS to MPL® in DETOX™PC is 10:1 (w/w). Each vial contains 300 μg CWS, 30 μg MPL®, 4.5 mg squalane, 0.6 mg TWEEN 80, 1.8 mg egg phosphatidylcholine and 60 μg α-tocopherol. Recommended storage of DETOX™PC is 2-8° C., and sterile water is used as a diluent. Preferably, 250 μg CWS and 25 μg MPL® is used per administration.

11D10 and 3H1 may be prepared using the same adjuvant if the two are to be used in the same preparation, or with the same or different adjuvants if the two are to be administered separately.

11D10 and 3H1 can be used in conjunction with other immunomodulators, such as, for example, interleukin 2 (IL-2), IL-4, IL-3, IL-12, GM-CSF, G-CSF, interferon and keyhole limpet hemocyanin (KLH).

11D10 and 3H1 can also be used in conjunction with other agents that serve to enhance and/or complement 3H1's and 11D10's effectiveness. Examples of such agents include, but are not limited to, peptides derived from CEA, HMFG, 3H1, or 11D10. Preferred HMFG and 11D10 peptides are those based on homology between 11D10and HMFG. Preferred CEA and 3H1 peptides are those based on homology between 3H1 and CEA.

Alternatively, 11D10 and/or 3H1 can be encapsulated in liposomes. Liposomes suitable for packaging polypeptides for delivery to cells are known in the art.

11D10 and/or 3H1 can be heat treated before administration and the heat treatment can be in the presence of adjuvant (as long as heat treatment does not compromise the activity of the adjuvant), for example, alum. If QS-21 is used, then the Ig portion of the adjuvant can be heated. Generally, DETOX™PC is not heated. For instance, 11D10 and/or 3H1 can be heated at about 40° to 80° C., preferably 45° C. to 60° C., for a period of about 5 minutes to 2 hours, preferably 15 minutes to 1 hour. Heat treatment is preferably at 45° C. for 30 minutes in a sterile vial in a water bath. The heat treatment can occur anytime before administration. Preferably, heat treatment is within 7 days of administration. Other heat treatment procedures can be used, as long as the desired activity of 11D10 and/or 3H1 is not significantly compromised. The heat-treated 11D10 and/or 3H1 are then administered as described herein.

For treatment using 11B10 and 3H1, effective amounts of 11D10 is and 3H1 are administered to an individual parenterally, preferably intracutaneously or subcutaneously. Other routes of administration include, but are not limited to, intramuscular and intradermal. If 11D10 and 3H1 are administered separately, the route of administration for the two antibodies may be the same or different. If alum (or aluminum hydroxide) precipitated 11D10 and/or 3H1 is used, the 11D10 and/or 3H1 is preferably administered intracutaneously. If QS-21 or DETOX™PC is used, 11D10 is the 3H1 and/or 11D10 is preferably administered subcutaneously. Depending on the particular adjuvant used, a manufacturer may provide suggested routes of administration as well as suggested amounts of adjuvants to be used. 11D10 and/or 3H1 can also be administered indirectly, by treatment of cultured cells followed by introduction of these cultured cells into an individual. The routes of administration can also vary during a course of treatment. For example, an individual can receive 11D10 and/or 3H1 intravenously followed by interperitoneal administration.

The amount of 11D10 given to the individual will depend upon several factors, such as the condition of the individual, the weight of the individual, the nature of the disorder or disease being treated, the extent of disease, the route of administration, how many doses will be administered, and the desired objective. Preferably, the dose per administration of 11D10 will range from about 10 μg to 20 mg, preferably 200 μg to 15 mg, more preferably 500 μg to 10 mg, even more preferably 1 mg to about 4 mg, even more preferably 2 mg. Preferably, the dose is 2 mg of alum-precipitated 11D10, 2 mg of 11D10 with QS-21, or 2 mg of 11D10 with DETOX™PC.

The amount of 3H1 given to the individual will depend upon several factors, such as the condition of the individual, the weight of the individual, the nature of the disorder or disease being treated, the extent of disease, the route of administration, how many doses will be administered, and the desired objective. Preferably, the dose per administration will range from about 10 μg to 20 mg, preferably 200 μg to 15 mg, more preferably 500 μg to 10 mg, even more preferably 1 mg to about 4 mg, even more preferably 2 mg. Preferably, the dose is 2 mg of alum-precipitated 3H1.

The interval between administrations of 11D10 and/or 3H1 can vary and will depend upon the disorder being treated and the responsiveness of the individual. Both 11D10 and 3H1, either in single or combined preparations, are preferably administered first as a priming dose followed by at least one, preferably two, more preferably three, boosting doses of either or both of 11D10 and 3H1. Further boosting doses may be given to enhance or rejuvenate the response on a periodic basis. 11D10 and/or 3H1 can be administered on a weekly, preferably biweekly (every two weeks), basis until a desired, measurable parameter is detected, such as elicitation of an immune response. Administration can then be continued on a less frequent basis, such as bimonthly (every two months) or monthly, as appropriate. Timing of subsequent injections (i.e., a maintenance dose) will depend, inter alia, upon the condition and response of the individual being treated. Ab3 levels for both antibodies can be monitored, preferably by the diagnostic methods described herein, to determine when maintenance (booster) administrations should be given, which could generally be about every two to three months. In one embodiment, the initial series of administrations is given at biweekly intervals for a total of four injections, followed by monthly injections.

It is understood that for some situations the individual receiving the 3H1/11D10 combination may be moderately to severely immunocompromised, either due to the nature of previous treatment, the disease itself, or both. Thus, the time required to mount an immune response and/or the number of injections of the 3H1/11D10 combination and/or the amount of 11D10 and/or 3H1 per administration may vary. For example, an individual may require a longer time to elicit an immune response once the 3H1/11D10 combination has been administered. In this case, it is recommended that the individual continue to be monitored for an immune response, even if no initial (i.e., within the first month) immune response has been detected. As another example, an individual may require more than the average number of injections to elicit an immune response. Alternatively, it may be desirable to have the intervals between injections longer than monthly for one or both of 11D10 and 3H1, for example, in order to optimize the immune response, such as a T cell response. Mounting an immune response is considered to be at least partially indicative, preferably completely indicative, of the effectiveness of the 11D10 and 3H1 combination in terms of obtaining beneficial or desired results and thus may be a useful indicator in determining effective amounts of 11D10 and 3H1.

One possible indication of effectiveness of administration of the anti-idiotype antibodies of the invention is the density of their respective TAA's on the tumor cells. To determine the effectiveness of 11D10, or whether administration of 11D10 is indicated, is the density of HMFG on the tumor cells may be used as an indicator. This density can vary widely from individual to individual, and may vary over the course of administration of 11D10 and/or over the course of the disease. As used herein, “density” of HMFG can refer to either or both of the following: (a) the number of cells per total cells in a given biological sample that have HMFG on their surface; (b) the amount of HFMG on the surface of each cell. Density (a) is calculated by noting the number of cells in a sample that are stained or otherwise indicate that HMFG is present divided by the total number of cells. This density would be preferably greater than about 20%, more preferably greater than about 30%, more preferably greater than about 50%, even more preferably greater than about 70%, even more preferably greater than about 80%, most preferably greater than about 90%. Thus, the invention includes administration of 11D10 to an individual having density of HMFG greater than about 20%, preferably greater than 30%, more preferably greater than 70%, even more preferably greater than about 80%, most preferably greater than about 90%.

Density (b) is indicated by the relative intensity of staining (or intensity of any measurement indicating the presence of HMFG) of cells in a sample from one individual relative to, for example, a sample from another individual. For this density, one skilled in the art can make an empirical determination of density. Density (b) is relative to normal tissues (i.e., cells lacking HMFG, or unaffected cells), so preferred ranges may be about 1.5 fold, preferably about 3 fold, more preferably about 10 fold higher expression over unaffected cells, as detected by immunohistochemical staining density. Unaffected cells could also be from the same individual.

This is not to say that individuals with lower densities, for example, less than about 50% are not indicated for administration of 11D10. While not wishing to be bound by a single theory, it is possible that administration of 11D10 could elicit a series of immuno-reactions that result in a more general response that is effective against an HMFG-associated tumor, such as a cytotoxic T cell response. A lower density, however, may indicate that additional therapies are desirable.

It is understood that density can also be used as an indicator of extent of disease and response to administration of 11D10. For example, a sample taken from an individual at the onset of 11D10 administration may exhibit about 80% density (i.e., about 80% of the cells exhibit HMFG). After receiving 11D10, a sample taken from the same location may exhibit only about 50% density, indicating that HMFG-expressing cells are being destroyed. Similarly, if the intensity of staining of a sample from an individual receiving 11D10 diminishes upon receiving 11D10, this indicates that HMFG-bearing tumor cells are being destroyed.

Similar tests of TAA as described herein may be used to determine the effectiveness of 3H1 administration, or whether 3H1 administration is indicated; in the case of 3H1, the TAA assessed is CEA. Such tests for CEA density are within the ordinary skill in the art. The initial densities of the two TAA's, HMFG and CEA, may be used to determine initial dosages of their respective anti-idiotype antibodies; subsequent densities may be used to determine dosages of the respective anti-idiotype antibodies in subsequent administrations. For the purpose of raising an immune response, 11D10 and/or 3H1 may be administered in an unmodified form. It may sometimes be preferable to modify 11D10 and/or 3H1 to improve its immunogenicity. As used herein, “immunogenicity” refers to a capability to elicit a specific antibody or cellular immune response, or both. Methods of improving immunogenicity include, inter alia, crosslinking with agents such as gluteraldehyde or bifunctional couplers, or attachment to a polyvalent platform molecule. Immunogenicity may also be improved by coupling to a protein carrier, particularly one that comprises T cell epitopes.

Administration of 11D10 can occur alone or in conjunction with other forms of therapy, whether established or experimental. “In conjunction with” means 11D10 can be given concurrently with, prior to, or after other therapies. For instance, 11D10 can be used to complement surgery, radiotherapy, chemotherapy and/or other drug therapies, either concomitantly or serially with respect to other therapies. The sequence and timing of these administrations can be determined empirically and will depend on such variables as the disease being treated, the condition of the patient, clinical history and indications, and/or responsiveness to various therapies. Such determinations are within the skill of the art. Administration of 11D10 can occur in conjunction with another anti-idiotype antibody. Use of 11D10 in conjunction with anti-idiotype antibody 3H1 has been discussed above. Administration of a combination of 11D10 and 3H1 can occur alone, or further in conjunction with other forms of therapy, whether established or experimental. For instance, a combination of 11D10 and 3H1 can be used to complement surgery, radiotherapy, chemotherapy and/or other drug therapies, either concomitantly or serially with respect to other therapies. The sequence and timing of these administrations, and the proper combination of 11D10 and 3H1 at each time, can be determined empirically and will depend on such variables as the disease being treated, the condition of the patient, clinical history and indications, and/or responsiveness to various therapies. Such determinations are within the skill of the art.

Preferably, 11D10 and 3H1 are administered before administration of other, adjunct therapies, such as chemotherapy and/or radiation, if these adjunct therapies are being used. Preferably, 11D10 is and 3H1 are administered 1 day, preferably 3 to 5 days, before the first course of chemotherapy and/or radiation therapy, and 1 day, preferably 3 to 5 days, prior to each cycle of chemotherapy and/or radiation therapy. This allows the individual more time to mount an immune response.

Administration of 11D10 and/or 3H1 can continue for various courses, depending on the individual and disease being treated. Preferably, administration of 11D10 and/or 3H1 is continued for as long as an individual is able to mount an immune response, whether humoral and/or cellular. Administration of 11D10 and/or 3H1 should be discontinued if the individual displays unacceptable adverse reactions that are associated with the administration of 11D10 and/or 3H1, and may or may not be continued if the individual displays progressive disease. Continuation of administration of 11D10 and/or 3H1 in the event of progressive disease depends at least in part on whether continued administration of 11D10 and/or 3H1 could supplement other indicated therapies.

Determining the Effects of Administration of 11D10 and 3H1

In order to determine the effect of administration of 11D10 and 3H1, an individual may be monitored for either an antibody (humoral) or cellular immune response against HMFG and CEA, or a combination thereof. The individual can also be monitored for disease progression.

Effects of 11D10 Administration

To determine the level of HMFG antibody (Ab3) in a biological sample, for example, serum or plasma is obtained from the individual. The sample may optionally be enriched for immunoglobulin before the assay is conducted, although this is not usually required. If a mouse immunoglobulin (such as 11D10) is to be used as an assay reagent, the sample is preferably pretreated to remove anti-mouse immunoglobulin activity. This may be performed, for example, by depletion on a mouse immunoglobulin column, or by mixing non-specific mouse immunoglobulin into the sample and removing any immunoprecipitate formed.

To conduct the assay, anti-HMFG that may be in the sample is contacted with a non-limiting amount of an antigenic equivalent of HMFG. This may be isolated HMFG, nitrocellulose with HMFG affixed by direct blotting or by transfer from a polyacrylamide gel, cells expressing HMFG (such as MCF-7 or SKBR3 cells which are human breast carcinoma cell lines), membrane preparations from such cells, or fixed tissue sections containing HMFG. Alternatively, an anti-idiotype, particularly 11D10, maybe used.

Once the immune complex has formed, it is generally separated from uncomplexed HMFG analog, and the amount of complex present is determined. The complex may be separated, for example, by centrifugation to collect cells or an immunoprecipitate, or capture by a solid phase. The amount of complex present may be measured by providing the HMFG analog with a label either directly, or by incubating with a secondary reagent. Alternatively, a competition assay may be performed, in which the sample is first incubated with the HMFG analog, and then a non-limiting amount of a labeled anti-HMFG reagent is added which competes with the anti-HMFG which may be present in the sample. Suitable labels include radiolabels, enzyme labels, fluorescent labels, and chemiluminescent labels. A standard curve is constructed using solutions known to contain no anti-HMFG, and solutions with various relative concentrations of anti-HMFG, in place of the sample. The sample containing the unknown amount of anti-HMFG is generally assayed in parallel, and the relative amount of anti-HMFG contained therein is determined by comparison with the standard curve. A preferred assay for determining anti-HMFG levels using HMFG antibody is radioimmunoassay (Example 2).

The isotype of the anti-HMFG antibody may be determined by including in the immunoassay an isotype-specific reagent(s), either at the separation or the labeling stage. For example, anti-human IgG may be used to separate or detect antibody of the IgG class present in a clinical sample of human origin. Presence of specific anti-HMFG of the IgG class generally indicates a memory response. Presence of anti-HMFG of the IgM class generally indicates ongoing immunostimulation, such as may be due to the presence of an HMFG expressing tumor, or ongoing treatment with 11D10.

If desired, anti-HMFG antibody detected in a biological sample may be further characterized; for example, by competition with anti-MC-10 (Ab1) to determine whether they are specific for related epitopes on HMFG. Competition assays between Ab1 and Ab3 are described in Example 2.

Anti-HMFG antibody may also be tested to determine whether it is cytotoxic. Complement mediated cytotoxicity (CMC) is determined, for example, by using HMFG-expressing target cells (such as MCF-7 or SKBR-3) labeled with ⁵¹Cr. Labeling may be accomplished by incubating about 10⁶ cells with ˜200 μCi Na₂⁵¹CrO₄ for 60 minutes at 37° C., followed by washing. The assay is conducted by incubating the antibody (or clinical sample containing the antibody) with the target cells. The opsonized cells are then washed and incubated with a source of complement; for example, guinea pig serum pre-adsorbed to remove intrinsic antibody activity. After a suitable incubation period at 37° C., release of ⁵¹Cr into the medium is determined and compared with that from unopsonized control cells. Release of ⁵¹Cr correlates with CMC activity.

Another way of characterizing the anti-HMFG antibody is by testing its ability to participate in an ADCC response (Cheresh et al. (1986) Cancer Res. 46:5112-5118). Radiolabeled HMFG-expressing target cells are incubated with the anti-HMFG (in the form of heat-inactivated serum), and effector cells. Normal human peripheral blood mononuclear cells (PBMC) are suitable effector cells, and preferably are used at an effector:target ratio of about 100. After approximately 4 hours at 37° C., the proportion of released ⁵¹Cr is determined as a measure of ADCC activity.

The cellular immune response in a subject being administered 11D10 may be quantified by conducting standard functional assays for specific T cell activity.

One type of assay measures T cell proliferation. In this test, peripheral blood mononuclear cells (PBMC) are obtained from a whole blood sample collected from the treated individual. For experimental animals, spleen cells may also be used. T cells may be enriched, for example, by centrifugation on a gradient such as FICOLL™. The cells are then cultured in the presence of 11D10 or HMFG or (more usually) irradiated HMFG expressing cells at various concentrations. Preferably, the stimulator cells are autologous with the responder cells, particularly in terms of histocompatibility Class II antigens. Extent of proliferation is then measured (often in terms of ³H-thymidine incorporation) in comparison to unstimulated cells. T cell proliferative activity in high risk patients' sera is shown in Example 2.

Another type of assay measures T cell cytotoxicity. In this test, an enriched T-cell population is used to effect lysis of ⁵¹Cr-labeled HMFG expression target cells, prepared as described above. Preferably, the effector cells are autologous with the target cells, particularly in terms of histocompatibility Class I antigens. The T cell population may optionally be pre-stimulated with HMFG or a relevant cell line. The T cells are then combined at various ratios with about 10⁴ labeled target cells; for example, in wells of a microtiter plate. The plate is optionally centrifuged to initiate cell contact, and the cells are cultured together for 4-16 hours at 37° C. The percent specific release of ⁵¹Cr into the medium is measured in comparison with labeled targets cultured alone (negative control) and targets lysed with a detergent such as 0.1% TRITON™ X-100 (positive control).

Other relevant measurements to determine the effect of 11D10 administration include clinical tests as may be appropriate in determining the development (i.e., progression) of cancer of the suspected type, whether direct or indirect indications of disease progression. Such tests may include blood tests, mammography, radioscintigraphy, CT scan, and MRI. Any measurable variable that correlates with disease progression is suitable. Any other tumor-associated marker is suitable for monitoring the course of therapy, such as, for example, carcinoembryonic antigen (CEA), or CA-125.

Effects of 3H1 Administration

To determine the level of CEA antibody (Ab3) in a biological sample, for example, serum or plasma is obtained from the individual. The sample may optionally be enriched for immunoglobulin before the assay is conducted, although this is not usually required. If a mouse immunoglobulin (such as 3H1) is to be used as an assay reagent, the sample is preferably pretreated to remove anti-mouse immunoglobulin activity. This may be performed, for example, by depletion on a mouse immunoglobulin column, or by mixing non-specific mouse immunoglobulin into the sample and removing any immunoprecipitate formed.

To conduct the assay, anti-CEA that may be in the sample is contacted with a non-limiting amount of an antigenic equivalent of CEA. This may be isolated CEA, nitrocellulose with CEA affixed by direct blotting or by transfer from a polyacrylamide gel, cells expressing CEA (such as LS174-T cells), membrane preparations from such cells, or fixed tissue sections containing CEA. Alternatively, an anti-idiotype, particularly 3H1, may be used.

Once the immune complex has formed, it is generally separated from uncomplexed CEA analog, and the amount of complex present is determined. The complex may be separated, for example, by centrifugation to collect cells or an immunoprecipitate, or capture by a solid phase. The amount of complex present may be measured by providing the CEA analog with a label either directly, or by incubating with a secondary reagent. Alternatively, a competition assay may be performed, in which the sample is first incubated with the CEA analog, and then a non-limiting amount of a labeled anti-CEA reagent is added which competes with the anti-CEA which may be present in the sample. Suitable labels include radiolabels, enzyme labels, fluorescent labels, and chemiluminescent labels. A standard curve is constructed using solutions known to contain no anti-CEA, and solutions with various relative concentrations of anti-CEA, in place of the sample. The sample containing the unknown amount of anti-CEA is generally assayed in parallel, and the relative amount of anti-CEA contained therein is determined by comparison with the standard curve. A preferred assay for determining anti-CEA levels using 3H1 antibody is radioimmunoassay.

The isotype of the anti-CEA antibody may be determined by including in the immunoassay an isotype-specific reagent(s), either at the separation or the labeling stage. For example, anti-human IgG may be used to separate or detect antibody of the IgG class present in a clinical sample of human origin. Presence of specific anti-CEA of the IgG class generally indicates a memory response. Presence of anti-CEA of the IgM class generally indicates ongoing immunostimulation, such as may be due to the presence of an CEA expressing tumor, or ongoing treatment with 3H1.

If desired, anti-CEA antibody detected in a biological sample may be further characterized; for example, by competition with anti-8019 (Ab1) to determine whether they are specific for related epitopes on CEA.

Anti-CEA antibody may also be tested to determine whether it is cytotoxic. Complement mediated cytotoxicity (CMC) is determined, for example, by using CEA-expressing target cells (such as LS174-T) labeled with ⁵¹Cr. Labeling may be accomplished by incubating about 10⁶ cells with ˜200 μCi Na₂⁵¹CrO₄ for 60 minutes at 37° C., followed by washing. The assay is conducted by incubating the antibody (or clinical sample containing the antibody) with the target cells. The opsonized cells are then washed and incubated with a source of complement; for example, guinea pig serum pre-adsorbed to remove intrinsic antibody activity. After a suitable incubation period at 37° C., release of ⁵¹Cr into the medium is determined and compared with that from unopsonized control cells. Release of ⁵¹Cr correlates with CMC activity.

Another way of characterizing the anti-CEA antibody is by testing its ability to participate in an ADCC response (Cheresh et al. (1986) Cancer Res. 46:5112). Radiolabeled CEA-expressing target cells are incubated with the anti-CEA (in the form of heat-inactivated serum), and effector cells. Normal human peripheral blood mononuclear cells (PBMC) are suitable effector cells, and preferably are used at an effector:target ratio of about 100. After approximately 4 hours at 37° C., the proportion of released ⁵¹Cr is determined as a measure of ADCC activity.

The cellular immune response in a subject being administered 3H1 may be quantified by conducting standard functional assays for specific T cell activity.

One type of assay measures T cell proliferation. In this test, peripheral blood mononuclear cells (PBMC) are obtained from a whole blood sample collected from the treated individual. For experimental animals, spleen cells may also be used. T cells may be enriched, for example, by centrifugation on a gradient such as FICOLL™. The cells are then cultured in the presence of CEA or (more usually) irradiated CEA expressing cells at various concentrations. Preferably, the stimulator cells are autologous with the responder cells, particularly in terms of histocompatibility Class II antigens. Extent of proliferation is then measured (often in terms of ³H-thymidine incorporation) in comparison to unstimulated cells.

Another type of assay measures T cell cytotoxicity. In this test, an enriched T-cell population is used to effect lysis of ⁵¹Cr-labeled CEA expression target cells, prepared as described above. Preferably, the effector cells are autologous with the target cells, particularly in terms of histocompatibility Class I antigens. The T cell population may optionally be pre-stimulated with CEA or a relevant cell line. The T cells are then combined at various ratios with about 10⁴ labeled target cells; for example, in wells of a microtiter plate. The plate is optionally centrifuged to initiate cell contact, and the cells are cultured together for 4-16 hours at 37° C. The percent specific release of ⁵¹Cr into the medium is measured in comparison with labeled targets cultured alone (negative control) and targets lysed with a detergent such as 0.1% Triton™ X-100 (positive control).

Other relevant measurements to determine the effect of 3H1 administration include clinical tests as may be appropriate in determining the development (i.e., progression) of cancer of the suspected type, whether direct or indirect indications of disease progression. Such tests may include blood tests, mammography, radioscintigraphy, CT scan, and MRI. Any measurable variable that correlates with disease progression is suitable. For instance, for CEA-associated tumors or disorders that are associated with measurable CEA in blood, CEA levels can be measured. Methods for measuring serum levels of CEA are known in the art and are commercially available as diagnostic kits (Hybritech Enzyme Immunoassay). For this test, serum is prepared as follows: Individuals treated with 3H1 will have 0.5 ml of serum treated with 1 ml of acetate buffer (pH 5.0) followed by heating at 90° C. for 15 minutes. After centrifugation at 2000 rpm for 10 minutes, the clear supernatant is tested for CEA using methods known in the art. The serum should be heat inactivated prior to testing as described because commercial CEA kits include a murine anti-CEA antibody and individuals receiving 3H1 usually have human mouse antibody (HAMA). Any other tumor-associated marker is suitable for monitoring the course of therapy, such as, for example, CA-125.

The invention also includes use of 11D10 and 3H1 for preparation of a medicament for use in treatment of HMFG and CEA-associated tumors, especially in those individuals with low tumor burden.

The following Examples are provided to illustrate but not limit the invention.

### EXAMPLES

### Example 1

### Production of 11D10 and 3H1 Anti-Idiotype Antibodies for Immunization

Production of 11D10

Murine monoclonal antibody MC-10 (recognizing a distinct epitope of HMFG) was used to immunize syngeneic BALB/c mice for the production of anti-idiotype antibody 11D10 (IgG1-κ) as described in commonly owned patent application Ser. No. 08/766,350 (attorney docket number 30414/2000321). Immunization of BALB/c mice, hybridoma fusion and cloning, selection of anti-idiotype (Ab2) and production of ascites in bulk quantities in mice were done as previously described. The Ab2 anti-idiotype 11D10 (IgG1) was purified from ascites by affinity chromatography on protein A-CL Sepharose 4B column followed by DEAE-Sepharose ion-exchange chromatography. The purity of the isolated immunoglobulin (>95%) was determined by sodium dodecylsulfate polyacrylamide gel electrophoresis (SDS-PAGE) and high pressure liquid chromatography techniques. Sterility, pyrogenicity, polynucleotides, mycoplasma and adventitious virus contamination and retrovirus removal validation tests were done in accordance with the United States Food and Drug Administration guidelines.

For use of alum-precipitated 11D10, 1 ml of 2% Alu-Gel S (Serva Fine Biochem, Inc., Garden City, Long Island, N.Y.) is added to 5 mg aliquots of purified mAb anti-Id (11D10). The volume is then adjusted to 10.0 ml with D-PBS and the mixture incubated on a vortex for one hour at room temperature. The mixture is then centrifuged at 2000 rpm at 24° C. for 10 minutes. The amount of mAb bound in the gel layer is determined by measuring spectrophotometrically the amount of unbound antibody in the supernatant. The Alu-Gel precipitated antibody is stored at 4° C. until use. These procedures are performed aseptically in a laminar flow hood and the final product was sterile and clearly labeled as anti-Id 11D10 Alu-Gel and aliquoted into pyrogen-free, sterile glass vials.

For use with QS-21 or DETOX(™)PC, 11D10 is vialed alone at 2 mg/ml into sterile, pyrogen-free vials.

Production of 3H1

Murine monoclonal antibody 8019 (recognizing a distinct epitope of CEA) was used to immunize syngeneic BALB/c mice for the production of anti-idiotype antibody 3H1 (IgG1-κ) as described in commonly owned patent application no. 08/579,940 (attorney docket number 30414/2000121). See also Bhattacharya-Chatterjee et al. (1987) J. Immunol. 5:562-573; Bhattacharya-Chatterjee et al. (1988) J. Immunol. 141:1398-1403. Immunization of BALB/c mice, hybridoma fusion and cloning, selection of anti-idiotype (Ab2) and production of ascites in bulk quantities in mice were done as previously described. The Ab2 anti-idiotype 3H1 (IgG1) was purified from ascites by affinity chromatography on protein A-CL Sepharose 4B column followed by DEAE-Sepharose ion-exchange chromatography. The purity of the isolated immunoglobulin (>95%) was determined by sodium dodecylsulfate polyacrylamide gel electrophoresis (SDS-PAGE) and high pressure liquid chromatography techniques. Sterility, pyrogenicity, polynucleotides, mycoplasma and adventitious virus contamination and retrovirus removal validation tests were done in accordance with the United States Food and Drug Administration guidelines.

To 5 mg aliquots of purified mAb anti-Id (3H1), 1 ml of 2% Alu-Gel S (Serva Fine Biochem, Inc., Garden City, Long Island, N.Y.) was added. The volume was then adjusted to 10.0 ml with D-PBS and the mixture incubated on a vortex for one hour at room temperature. The mixture was then centrifuged at 2000 rpm at 24° C. for 10 minutes. The amount of mAb bound in the gel layer was determined by measuring spectrophotometrically the amount of unbound antibody in the supernatant. The Alu-Gel precipitated antibody was stored at 4° C. until use. These procedures were performed aseptically in a laminar flow hood and the final product was sterile and clearly labeled as anti-Id 3H1 Alu-Gel and aliquoted into pyrogen-free, sterile glass vials.

### Example 2

### Use of 11D11 to Treat High Risk Individuals in the Adjuvant Setting

Selection of Patients

High risk patients with HMFG positive tumors are selected for this study. These patients do not have advanced disease, i.e., do not have detectable metastases. Generally, patients have received adjuvant chemotherapy and/or radiation therapy for breast cancer, non-small cell lung cancer, or ovarian. Those patients usually receive 11D10 at completion of treatment (typically at least 4 weeks after completion of treatment). Patients on hormone therapy receive 11D10 concurrently with treatment. Thus far, 4 patients have accrued to this study. Baseline studies include complete physical examination, chest radiography, computer axial tomography examination of the abdomen, routine blood counts and chemistries.

Preparation of Ab2

11D10 is obtained and alum-precipitated or mixed with QS-21 or DETOXPC as described in Example 1. The final product is tested for sterility, pyrogenicity and general safety in guinea pigs before use. An Investigational New Drug Application was approved through the United States Food and Drug Administration(BB-IND 5745). Before administration, 11D10 is heat treated in the presence of adjuvant at 45° C. for 30 minutes in a water bath. If alum is the adjuvant, 11D10 is heat treated in the presence of alum.

Treatment Schedule

All patients receive 2 mg 11D10 with adjuvant. Patients enter one of three regimens: (a) 2 mg of aluminum hydroxide (alum) precipitated 11D10; (b) 2 mg of 11D10 mixed with 100 g QS-21; (c) 2 mg of 11D10 mixed with DETOX (250 g CWS+25 g MPL®). For regimen (c), 1.08 ml of 11D10 solution is mixed with 0.12 ml DETOX PC, and 1.0 ml is withdrawn for injection.

Injections are intracutaneous if aluminum hydroxide-precipitated 11D10 is used. Injections are subcutaneous if QS-21 or DETOX™PC is used. Four injections are given every two weeks, followed by monthly injections for a total of 24 months as long as there is immunological response and no evidence of progressive disease. Patients are evaluated every 12 weeks. Patients are removed from this study if they demonstrate progressive disease.

Toxicity and Responses

Toxicity is monitored for each patient, including analysis of hematopoietic cells, renal function, and hepatic function. Patients are also monitored very closely for disease activity.

Assays for Humoral Immunity

(a) Total anti-11D10 Response

The development of humoral immunity induced by immunization with 11D10 is assessed by testing sera obtained from patients before therapy and after each treatment with the vaccine. The sera is initially tested for total human anti-murine-antibody responses including anti-iso/allo/and anti-anti-idiotype antibodies by sandwich radioimmunoassay as described by Khajaeli et al. (1988) J. Nat'l Cancer Inst. 80:937-942. Briefly, microtiter plates are coated with 11D10 and incubated with different dilutions of patients' sera. After washings, the antigen-antibody reaction was tagged using ¹²⁵I-labeled anti-Id 11D10 in a homogeneous sandwich radioimmunoassay. Since 11D10 is injected as intact IgG1, patients are expected to mount human anti-mouse antibody responses.

(b) Specific Ab3 Response to Ab2

Sera from immunized patients with positive HAMA responses are tested for the presence of anti-antiidiotypic antibodies as follows. Sera are preincubated with normal murine immunoglobulin to block human antibodies against isotype and allotypic determinants and then checked for the presence of anti-anti-Id (Ab3) by reaction with 11D10 coated onto microtiter plates by RIA. Unrelated Ab2 serves as a control. After washing, the antigen-antibody reaction is tagged using ¹²⁵I-labeled 11D10 in a homogeneous sandwich RIA as described above. Pretreatment non-immune sera and sera from normal donors serve as controls.

If a positive reaction is obtained, the sera are checked for the ability to inhibit the binding of ¹²⁵I-labeled Ab1 (MC-10) to Ab2 (11D10) on the plate by radioimmunoassay or vice versa (inhibition of radiolabeled Ab2 binding to Ab1 on the plate). These reactions are done in the presence of excess normal murine immunoglobulin to block human antibodies against isotypic and allotypic determinants.

(c) Binding of Ab3 to Tumor Antigen

To assess humoral immune responses directed against native target antigens, patients' Ab3 sera is tested for reactivity with cell lines known to express MC-10 antigen such as MCF-7 cells in an RIA and also by FACS analysis. MCF-7 cells are available from the ATCC. In addition, the sera are checked for reactivity against a solubilized semi-purified preparation of MC-10 antigen (i.e., HMFG) and coated onto microtiter plates. The antigen-antibody reaction is detected by using ¹²⁵I-labeled anti-human Ig reagents. Pre-immune sera is used as a control. Unrelated antigen is also used in the assay. Isotype of human Ab3 sera binding of MC-10 antigen is determined by ELISA using anti-human isotype specific reagents.

(d) Epitope Analysis of Ab3

To demonstrate that Ab3 generated in treated patients and Ab 1 (MC-10) bind to the same antigenic determinant, inhibition of MC-10 binding to Ag positive tumor cell line or MC-10 antigen by Ab3 sera is checked by RIA. A fixed amount of radiolabeled MC-10 (˜90,000 cpm) is co-incubated with different concentrations of patients' purified Ab3 or Ab1 preparations and MCF-7 cells.

Ab3 is purified from patients' sera as follows. Fifteen milliliters of hyperimmune serum are passed over an immunoadsorbent column consisting of immunizing anti-idiotype immunoglobulin (11D10) coupled to Sepharose 4B. Anti-anti-idiotypic antibodies (Ab3) bound to the column are eluted with 0.1 M glycine-hydrochloric acid buffer (pH 2.4). The eluted antibody is neutralized with 3M Tris, dialyzed against PBS, pH 7.2 and then passed over an immunoadsorbent column consisting of allotype matched normal mouse immunoglobulin coupled to Sepharose 4B to remove anti-isotypic and anti-allotypic reactivities. Antibody that passes through is concentrated and used as purified Ab3. The isotype of Ab3 is determined by ELISA using human anti-isotype specific reagents (Tago).

Inhibition curves obtained with Ab1 and Ab3 that are very similar at different dilutions indicates that the patients' Ab3 binds to the same antigenic epitope as Ab1 and therefore contains antibody molecules with Ab1 properties.

(e) Cytotoxic Activities of Ab3

If Ab3 in patients' sera bind specifically to tumor cells, the ability of Ab3 to lyse these cell in conjunction with effector cells and/or complement is tested by standard ADCC (Cheresh et al. (1986)) or CMC assays, (Herlyn et al. (1981) Int. J. Cancer 27:769). However, cytotoxic activity of the Ab3 may be dependent on its isotype, IgG1 being effective in ADCC and IgG1, IgG2, IgG3 and IgM in CMC.

Patients' sera are tested for ability to mediate antibody dependent cellular cytotoxicity (ADCC). Cheresh et al. (1986). For this assay, cultured human MCF-7 cells (which express HMFG on the cell surface) are used as target cells and were labelled with ⁵¹Cr. Normal human peripheral blood mononuclear cells (PBMC) are used as effector cells. The ADCC assay is performed in the presence of heat inactivated patient's serum with an effector to target cell ratio or 100:1 for 4 hours, followed by measurement of amount of ⁵¹Cr released.

(f) Quantitation of the Ab3 and Ab1 Response

The expression of anti-anti-Id antibody (Ab3) in the patients' sera is quantitated by RIA inhibition studies as follows. Briefly, microtiter plates are coated with MC-10 IgG1 (Ab1) and reacted with a fixed amount of ¹²⁵I-labeled 11D10. A standard inhibition curve is generated using purified MC-10 IgG1 as inhibitors. Next, patients' sera depleted of anti-iso-allotypic activity at different dilutions is checked for its ability to inhibit the Ab1-Ab2 reaction and the amount of Ab1-like antibody in the sera is estimated from the standard inhibition curve. The induction of Ab3 response as well as duration is compared among different adjuvants. If there is no statistical difference between Ab3 responses or duration at a number of doses, the titer of specific anti-tumor response (Ab1′) in the sera by ELISA assay is compared against semi-purified MC-10 antigen coated plates.

(g) In Vitro Studies

If circulating Ab1′ is not detected in Ab3 positive patients' sera, that may indicate that they may be bound to patients' tumor cells, or to circulating tumor antigen or they are of low affinity. These patients' PBMC are stimulated in vitro with antigen or Ab2 for the induction of tumor specific antibody. For this, PBMC obtained from blood collected before therapy, every three months, one month after the last immunization, and three months after the last immunization is cultured with various concentrations of 11D10, or unrelated Ab2, or MC-10 antigen (10 μg to 100 ng) in a modified Mishell-Dutton culture. Culture supernatants are harvested and checked first for the production of specific human immunoglobulins by ELISA assay and for binding to insolubilized preparation of Ab2 by radioimmunoassay. In addition, the supernatants are tested for the content of idiotope bearing molecule by their ability to inhibit the reaction between the ¹²⁵I-labeled MC-10 (Ab1) to 11D10. The supernatants are also checked for their reactivity with MC-10 Ag-positive MCF-7 cells and Ag-negative cells such as M21/P6 or MOLT-4 in a binding assay with ¹²⁵I-labeled anti-human Ig reagents by RIA or ELISA assay (sensitivity>1 ng) for the evaluation of Ab1′ antibody.

The specificity of the effect of 11D10 is monitored by incubating PBMC with unrelated Ab2 of the same isotype. Since only Ab3 positive patients will be included in this in vitro study, PBMC stimulated with 11D10 should secrete antibodies binding to 11D10 and serve as a positive control.

Assays for Cell-Mediated Immunity

The goal is to examine whether a specific T cell response to the tumor associated MC-10 antigen is generated in patients with HMFG-associated tumors, particularly breast cancer, following a series of immunizations with the anti-idiotype antibody 11D10 in alum or mixed with QS-21 or with DETOX™PC. Immunization with the vaccine could result in the generation of antibodies which alone can block T cell function. Nevertheless, considering the importance of T cells in the anti-tumor response, particularly CTL, it is necessary to examine whether this immunologic function exists.

T cell-mediated immunity is checked by: 1) testing if a T cell response is present which targets MC-10 antigen on the tumor cells, and 2) testing whether this response increases with repeated immunizations. The analysis proceeds in two phases. The first phase is to determine whether T cells from all PBMC samples received can be specifically expanded following in vitro immunizations against the 11D10 anti-Id antibody. If this occurs, it is determined whether these T cells can lyse or release cytokines against autologous MC-10 antigen bearing breast tumor cells and/or allogeneic MC-10 antigen expressing cancer cells sharing a single class I HLA antigen in common with the autologous CTL.

All patients entered into the trial undergo phlebotomy to collect one unit of whole blood prior to the first immunization. PBMC are isolated by standard Ficoll-Hypaque separation and cryopreserved for all future studies. These PBMC provide 1) antigen presenting feeder cells for subsequent studies, and 2) serve as baseline for T cell responses. In addition, following each immunization, 60 ml of peripheral blood is drawn, Ficoll-Hypaque separated and cryopreserved for the determination of T cell responses.

The T cell responses studied are generation of specific cytotoxic and/or cytokine producing T cells and proliferation of the T cell cultures in response to the antigens. When available, lymph node biopsies are obtained from the patients to provide a source of tumor infiltrating lymphocytes (TIL). Similar studies are conducted where possible using TIL to determine if tumor biopsies become a source of MC-10 antigen specific cells. Khazaeli et al. (1988) J Natl. Cancer Inst. 80:937-942; Cheresh et al. (1985) Proc. Natl. Acad. Sci. U.S.A. 82:515. Also, tumor biopsies provide a source of tumor cells to serve as critical autologous targets for cytotoxicity assays, cytokine production, and proliferation assays.

(a) In vitro Functional Activity of T Cells

Ficoll-Hypaque separated PBMC (1-3×10⁶) is incubated in the presence of: IL-2 alone (10 Cetus units/ml), 0.1 to 100 μg/ml anti-Id 11D10 antibody or HMFG. The cell culture medium consists of Iscoves medium supplemented with 10% human AB serum, gentamycin, sodium pyruvate, non-essential amino acids, L-glutamine and 10 Cetus units/ml recombinant IL-2. Every seven days the cultures are stimulated with irradiated autologous PBL pre-sensitized with the appropriate antigen used by day 0. The methods of in vitro sensitization are similar to those recently described (Steven et al. (1995) J. Immunol. 154:762). Beginning day 21 and repeated on a weekly basis, proliferating cells are assessed for cell surface phenotype and cytotoxic and cytokine producing potential. Initially, all T cells are tested for their ability to recognize and lyse in 4 hours ⁵¹Cr release assays autologous EBV cells alone and autologous EBV transfected B cells with the cDNA containing the sequence for the 11D10 anti-Id molecule. Cultures lysing 11D10 transfected autologous EBV cells>10% are further tested against the NK sensitive line K562, the LAK sensitive line Daudi, autologous tumor if available and other HLA matched and mismatched HMFG bearing breast tumor cells. In addition, GM-CSF is assayed to determine if there is specific release of cytokines in addition to or in place of specific cytotoxicity. Proliferation of the cultures to the agents is determined by increases in cell numbers following in vitro stimulations.

Survival Results

Survival data is calculated based on length of time a patient has no detectable disease (i.e., length of time until progression). This length of time is determined based on entry date into the study. An even more meaningful statistic is length of time of no detectable disease (or to progression) as measured from the date of the last treatment.

### Initial Data From Study

The study described in this Example was initiated. This data represents results as of May, 1998, from a total of 14 patients enrolled in the study. Of the 14 patients, 11 are currently receiving 11D10. Of the 3 patients who are off the study, 2 were withdrawn after progressive disease (the other patient withdrew from the study). Six of the patients receive QS-21; 3 of the patients receive alum (adjuvant data for the remaining 2 patients was not available).

Of 10 patients tested for antibody response, all 10 showed production of Ab3. All 7 patients tested for T cell proliferation showed an increase over baseline upon administration of 11D10 antibody, with various patterns of levels subsequent to this initial increase.

Six patients have been on the study for over 300 days, with two of those patients on the study over 400 days. For the two patients who showed progressive disease, time to progression was 92 days for one patient and 119 days for the other patient (both of these patients were receiving QS-21). There have been no deaths.

### Example 3

### Use of 11D10 to Treat Individuals with Low Tumor Burden

An individual who has been diagnosed as having an HMFG-associated tumor, such as breast cancer, is assessed for extent of disease using standard diagnostic imaging techniques such as CT scan. If the assessment shows that the individual does not have advanced disease, preferably no lymph node involvement, then the individual is given 11D10 in the same regimen as in Example 2. The individual is monitored for an immune response (see Example 2) and for extent of disease. Treatment is continued as long as an immune response is maintained, even if the disease becomes undetectable by the methods described herein. Intervals between administration of 11D10 may increase (i.e., longer than monthly) as long as an immune response is maintained and disease does not appear to progress.

### Example 4

### Administration of 11D10 to an Individual at Risk for Developing HMFG-Associated Tumor or Having Residual Disease

An individual who is adjudged to be at risk for developing HMFG-associated tumor due to, for example, family history of HMFG-associated tumors, is administered 11D10 bi-weekly (or as often as twice a week) until an immune response is observed (see Example 2). Upon elicitation of an immune response, the interval between 11D10 administrations is increased by one week for each administration until the immune response begins to decrease. The interval between administrations of 11D10 is then sequentially adjusted to the previous interval until the immune response remains constant (i.e., is no longer decreasing). Administration of 11D10 is maintained at that interval. The individual is monitored for disease development every one to two years.

As a more particular example of this procedure, a 33 year-old woman elects to begin administration of 11D10 based on her family history of HMFG-associated breast cancer (mother, grandmother, and aunt had developed the disease). Injections begin on a bi-weekly basis until an immune response is detected (usually one to four months). The next injection is given after one week. The following injections are given as follows: (a) after two weeks, then (b) after three weeks, then (c) after four weeks, then (d) after five weeks, then (e) after six weeks, then (f) after seven weeks, then (g) after eight weeks. Injections are maintained every two months while monitoring the immune response every month. If the immune response is constant, the injections are given as follows: (a) every 9 weeks, then (b) every 10 weeks, then (c) every 11 weeks, then (d) every 12 weeks. Injections are maintained every three months while monitoring the immune response. If the immune response is constant, the intervals between 11D10 injections are increased by one week until injections are given every 6 months. If the immune response declines, then the interval is shortened until the response is regained to its original level. The individual is maintained on 11D10 administrations during her lifetime. If HMFG-associated tumors develop, then other therapies may be administered in conjunction with, or in lieu of, 11D10.

As another particular example, an individual with HMFG-associated breast cancer has the tumor resected, and there is no known lymph node involvement. No disease is detectable after surgery. Administration of 11D10 commences and is adjusted as described above, and the individual is monitored for disease progression.

### Example 5

### Administration of 11D10 in the Neo-Adjuvant Setting

An individual who has been diagnosed with an HMFG-associated tumor, such as an HMFG-associated breast or ovarian cancer, is scheduled to obtain treatment such as surgery and/or chemotherapy. During the time between diagnosis and the initiation of treatment (i.e., while the patient is waiting for these treatment(s) to commence), 11D10 is administered as described in Example 2. Administration of 11D10 continues after commencement of these treatment(s) and after the course of these treatment(s). The interval between administration of 11D10 is adjusted to that the individual maintains an immune response.

### Example 6

### Use of 11D10 and 3H1 to Treat Individuals with Low Tumor Burden

Two individuals who have been diagnosed as having breast cancer, but have no metastatic (advanced) disease were administered 2 mg each of 11 D10 and 3H1, both prepared in Alugel. In each round of administration, subcutaneous injections of 11D10 and 3H1 were given at separate sites, for example 11D10 on one arm and 3H1 on the leg. Four administrations were given every two weeks, followed by monthly injections. One individual was administered both antibodies over a period of about 25 months. Another individual was administered both antibodies over a period of about 21 months. Serum samples were obtained from each individual prior to the first administration, and following certain subsequent administrations. Serum samples were analyzed for immune response to 11D10 and 3H1 by all or some of the following assays:

(i) Sandwich assay (HAMA): Plates were coated with either 11D10 (to assay immune response to 11D10 administration) or 3H1 (to assay immune response to 3H1 administration) (5 μg/ml, 50 μl/well) and incubated overnight at room temperature. After that, plates were blocked with 1% bovine serum albumin (BSA) in phosphate buffered saline (PBS) for 1 hour. Then different dilutions of treated inviduals' sera were added and incubated for 2 hours at room temperature. The plates were then washed with PBS and either ¹²⁵I-11D10 (50 μl/well, ˜90,000 cpm) or ¹²⁵I-3H1 (50 μl/well, ˜90,000 cpm) was added and incubated for 1.5 hours. After washing, the wells were counted in a Gamma counter.

(ii) Inhibition Assay-1: Plates were coated with MC10 (to assay immune response to 11D10 administration) or 8019 (to assay immune response to 3H1 administration) (5 μg/ml, 100 μg/ml) and were incubated overnight at RT. The plates were then blocked with 1% BSA in PBS. Then different dilutions of patients' sera along with ¹²⁵I-11D10 or ¹²⁵I-3H1 (50 μg/well, ˜90,000 cpm) were added and incubated for 2 hrs at RT. After washing, the wells were counted in Gamma counter. The percentage of inhibition of the assay was calculated according to the formula:

% Inhibition=

$$1 - [ \frac{{R}_{T} - {R}_{C}}{{R}_{max} - {R}_{C}} ] \times 100$$

in which R_{T} was the average cpm of the experimental well with inhibitors, Rc was the average background cpm and Rₘₐₓ was the average maximum binding without inhibitors.

(iii) Inhibition Assay-2: Plates were coated with 11D10 (to assay response to 11D10 administration) or 3H1 (to assay response to 3H1 administration) (5 μg/ml, 100 μg/ml) and were incubated overnight at RT. The plates were then blocked with 1% BSA in PBS. Then different dilutions of patients' sera along with ¹²⁵I-MC10 (50 μg/well, ˜90,000 cpm) or ¹²⁵I-8019 were added and incubated for 2 hrs at RT. After washing, the wells were counted in Gamma counter. The percentage of inhibition of the assay was calculated according to the formula:

% Inhibition=

$$1 - [ \frac{{R}_{T} - {R}_{C}}{{R}_{max} - {R}_{C}} ] \times 100$$

in which R_{T} was the average cpm of the experimental well with inhibitors, Rc was the average background cpm and Rₘₐₓ was the average maximum binding without inhibitors.

(iv) T Cell Proliferation Assay: Peripheral blood mononuclear cells were isolated by standard Ficoll-Hypaque density gradient centrifugation method and 2×10⁵ cells per well were incubated with different concentrations of 11D10 Alugel and 11D10 IgG, 4DC6 Alugel and 1A7 (2 μg, 1 μg, 10 ng, 100 ng) (to assay immune response to 11D10 administration) or with different concentrations of 3H1 Alugel and controls 4DC6 Alugel, 4DC6 (2 μg, 1 μg, 10 ng, 100 ng) (to assay immune response to 3H1 administration) in RPMI medium with 5% normal pooled human serum (AB donor heat inactivated, Pel-Freeze, WI, Code# 34004-1). The non-specific mitogen phytohemaglutinin P was used as a positive control at 5, 2 and 1 μg per well. After the cells were incubated for 5 days at 37° C. in an atmosphere containing 5% CO₂, they were pulsed with [³H] thymidine (1 uCi/well for 20 hrs). Data were expressed as mean counts (triplicate wells per minute of thymidine incorporation).

T cell Proliferation Index (PI) was calculated according to the formula:

$$PI = \frac{CPM in antigen stimulated wells}{CPM in medium stimulated wells}$$

(v) CEA binding inhibition assay: Plates were coated with CEA (2 μg/ml, 100 μg/ml, 100 μg/well) and were incubated overnight at room temperature (RT). The plates were then blocked with 1% BSA in PBS. Then different dilutions of patients' sera along with ¹²⁵I-8019 (50 μg/well, ˜90,000 cpm) were added and incubated for 2 hrs at RT. After washing, the wells were counted in gamma counter. The percentage of inhibition of the assay was calculated according to the formula:

% Inhibition=

$$1 - [ \frac{{R}_{T} - {R}_{C}}{{R}_{max} - {R}_{C}} ] \times 100$$

in which R_{T} was the average cpm of the experimental well with inhibitors, Rc was the average background cpm and Rₘₐₓ was the average maximum binding without inhibitors.

Data obtained for Patient 1 are as follows:

Immune response to 11D10 administration

| No. of Administrations | | | | | | | | | |
|---|---|---|---|---|---|---|---|---|---|
| Test | Units | Pre | 5 | 8 | 11 | 16 | 20 | 25 | 28 |
| Sandwich Assay | cpm | 873 | 5681 | 66948 | 36066 | 39031 | 58151 | 48550 | 45996 |
| (1:10) | | | | | | | | | |
| Inhibition 1 | % Inhi | 0 | 59 | — | — | — | — | — | — |
| Ab1 + Ab3 + ¹²⁵I − Ab2 | | | | | | | | | |
| Ab3 (anti-anti-id) | | | | | | | | | |
| Inhibition 2 | % Inhi | 4 | 75 | 60 | 61 | 63 | 84 | 89 | 87 |
| Ab2 + Ab3 + ¹²⁵I − Ab1 | | | | | | | | | |
| Ab3 (anti-anti-id) | | | | | | | | | |
| T-cell Proliferation | S.I. | 1.0 | 1.6 | 1.4 | 42.8 | — | 131.8 | 10.23 | 1.19 |
| Assay | | | | | | | | | |
| (100 ng 11D10-Alu) | | | | | | | | | |

Immune response to 3H1 Administration

| No. of Administrations | | | | | | | | | |
|---|---|---|---|---|---|---|---|---|---|
| Test | Units | Pre | 5 | 8 | 11 | 16 | 20 | 25 | 28 |
| Anti-3H1 | cpm | 985 | 3945 | 8306 | | 42830 | 41512 | 43870 | B64926 |
| HAMA | | | | | | | | | |
| (1:10) | | | | | | | | | |
| Inihition 1 | % Inhi | 0 | 59 | — | — | 70 | 80 | 89 | 89 |
| Inihibition 2 | % Inhi | 32 | 50 | 31 | — | 50 | 78 | 87 | 88 |
| CEA | % Inhi | 0 | 22 | — | — | 92 | 88 | 85 | 90 |
| Binding | | | | | | | | | |
| Inhibition | | | | | | | | | |
| T-cell | S.I. | — | — | 4.7 | 66.2 | — | 13.62 | 4.22 | 1.38 |
| Proliferation | | | | | | | | | |
| Assay | | | | | | | | | |
| CEA T-Cell | S.I. | — | — | — | 4.1 | — | 2.36 | — | — |
| | Peptide | | | | | | | | |

Data obtained for patient 2 are as follow:

Immune response to 11D10 Administration

| No. of Administrations | | | | | | | | | |
|---|---|---|---|---|---|---|---|---|---|
| Test | Units | Pre | 5 | 8 | 11 | 17 | 20 | 23 | 26 |
| Sandwich Assay | cpm | 551 | 1135 | 10603 | 14536 | 24860 | 34522 | 34397 | 38451 |
| (1:10) | | | | | | | | | |
| Inhibition 1 | % Inhi | 4 | 20 | — | — | — | — | — | — |
| Ab1 + Ab3 + ¹²⁵I − Ab2 | | | | | | | | | |
| Ab3 (anti-anti-id) | | | | | | | | | |
| Inhibition 2 | % Inhi | 4 | 24 | 60 | 42 | 64 | 78 | 88 | 90 |
| Ab2 + Ab3 + ¹²⁵I − Ab1 | | | | | | | | | |
| Ab3 (anti-anti-id) | | | | | | | | | |
| T-cell Proliferation | S.I. | 2.6 | 24.5 | — | 23.2 | 1.4 | 38.2 | 96.06 | 123.20 |
| Assay | | | | | | | | | |
| (100 ng 11D10-Alu) | | | | | | | | | |

Immune response to 3H1 Administration

| No. of Administrations | | | | | | | | | |
|---|---|---|---|---|---|---|---|---|---|
| Test | Units | Pre | 5 | 8 | 11 | 17 | 20 | 23 | 26 |
| Anti-3H1 | cpm | 952 | 1116 | 3941 | 23546 | 26312 | 22604 | 35910 | 39549 |
| HAMA | | | | | | | | | |
| (1:10) | | | | | | | | | |
| Inihition 1 | % Inhi | 0 | 33 | 77 | 68 | 48 | 70 | 83 | 85 |
| Inihibition 2 | % Inhi | 18 | 29 | 57 | 59 | 34 | 60 | 75 | 80 |
| CEA Binding | % Inhi | 0 | 28 | 33 | 72 | 59 | 78 | 67 | 73 |
| Inhibition | | | | | | | | | |
| T-cell | S.I. | — | — | — | — | 1.46 | 6.45 | 16.99 | 30.54 |
| Proliferation | | | | | | | | | |
| Assay | | | | | | | | | |
| CEA T-Cell | S.I. | | | | | 3.00 | 1.97 | 2.37 | ND |
| | Peptide | | | | | | | | |

The data show that neither patient presented with clinical complications due to administration of both 11D10 and 3H1. Both patients generated active immunity to HMFG and CEA.

### Example 7

### Use of 11D10 and 3H1 In Combination with Chemotherapy

Selection of Patients

Patients with HMFG- and CEA-positive tumors, generally breast cancers (including metastatic breast cancer), are selected for this study. These patients generally show histological, cytological, or radiologic evidence of metastatic breast carcinoma excepting inflammatory breast cancer. These patients also generally are to receive an anthracycline- or taxane-based chemotherapy regimen as first-line therapy for metastatic disease. Patients may have had either i) previous surgery and radiotherapy, ii) adjuvant/neoadjuvant chemotherapy, iii) adjuvant and/or palliative hormonal therapy. Baseline studies may include complete physical examination, chest radiography, computer axial tomography examination of the abdomen, routine blood counts and chemistries.

Dose, Administration and Treatment Schedule

11D10 is provided adsorbed to aluminum hydroxide at a concentration of 2 mg/ml. 3H1 is also provided adsorbed to aluminum hydroxide at a concentration of 2 mg/ml. Patients receive 11D10 and 3H1 every two weeks for 4 doses and then every 28 days thereafter in combination with their first-line chemotherapy. In this study, 11D10 and 3H1 are administered as 2 mg intradermal (ID) injections every other week for 4 doses followed by subcutaneous (SC) maintenance injections every 28 days. The first cycle of chemotherapy begins after the patient has received 2 doses of 11D10 and 3H1. Chemotherapy begins at the start of Week 3. 11D10 and 3H1 are administered prior to chemotherapy administration on days when chemotherapy administration and 11D10/3H1 administration visits occur on the same day. First-line therapy can consist of any anthracycline- or taxane-based chemotherapy with or without Herceptin.

Once the first four doses of 11D10 and 3H1 have been administered, the schedule of events for 11D10 and 3H1 (biweekly 11D10 and 3H1 for 4 injections, then every other 4 weeks for 19 months) is followed regardless of chemotherapy frequency, or changes in regimen. 11D10 is generally administered as 1 ml volume of 11D10 2 mg (11D10 aluminum hydroxide-precipitated anti-idiotype monoclonal antibody) in preservative-free saline and 3H1 is generally administered as 1 ml volume of 3H1 2 mg (3H1 aluminum hydroxide-precipitated anti-idiotype monoclonal antibody) in preservative-free saline.

Patients continue on 11D10 and 3H1 for up to nineteen (19) months and are then be followed for survival. Patients may continue to receive chemotherapy or second line therapy as deemed appropriate by their treating physicians. Patients may continue to receive 11D10 and 3H1 after discontinuation of chemotherapy or second line therapy, in the event of diminished performance status or disease progression.

Assays for Immune Response

Blood samples are collected at baseline, week 14, week 30, week 54, week 78 and week 82 in order to evaluate immune response and other surrogate markers of clinical benefit. Various assays can be used to determine immune response to administration of 11D10 and 3H1, including assays described in the preceding Examples, and HAMA, Ab3, anti-CEA and anti-HMFG antibody response. Cellular assays (T-cell proliferation and reverse Elispot) can also be conducted.

(i) HAMA: This assay measures the level of human anti-mouse antibodies (HAMA), which are endogenous antibodies against mouse immunoglobulin. Since 11D10 and 3H1 are injected as intact murine IgG1, patients are expected to mount human anti-mouse antibody responses.

(ii) Ab3 Assay (anti-anti-Id): This assay would demonstrate whether Ab3 generated in patients with a positive Ab3 response share idiotypes with Ab1 (mAb BrE-1 or 8019). This non-specific idiotype assay is an inhibition assay with a positive response defined as >25% inhibition of Ab1 (BrE-1 or 8019) binding to Ab2 (11D10 or 3H1) in patients' sera following adequate immunization (minimum of six 11D10+3H1 immunizations).

(iii) Anti-HMFG and -CEA Antibody Assays: Direct, inhibition and competitive assays are used to demonstrate whether Ab3 bind specifically to the primary antigen, HMFG and CEA, used as a purified target in ELISA.

(iv) T-Cell Proliferation Assay: This assay would demonstrate whether patients' PBMCs can be specifically stimulated to proliferate by HMFG and CEA or by Ab2.

(v) Reverse ELISPOT Assay: The assay would demonstrate whether PBMCs can be specifically stimulated by CEA and HMFG or Ab2 to produce IL-2 or IFN gamma.

Survival Results

Survival data is calculated based on length of time a patient has no detectable disease (i.e., length of time until progression). This length of time is determined based on entry date into the study. An even more meaningful statistic is length of time of no detectable disease (or to progression) as measured from the date of the last treatment. Disease progression can also be measured based on progression in the disease as indicated by staging markers for the particular cancer type.

### Example 8

### Use of 11D10 and 3H1 In Combination with First Line Hormonal Therapy

Selection of Patients

Patients with HMFG- and CEA-positive tumors, generally breast cancers (including metastatic breast carcinoma), are selected for this study. These patients generally show histological, cytological, or radiologic evidence of metastatic breast carcinoma (measurable or evaluable). These patients also generally are to receive one of the following hormonal therapies as first-line therapy for metastatic disease: Femara, Aridimex, Aromasin or Tamoxifen. Generally, their estrogen receptor and/or progesterone receptor status is either positive or unknown. Patients may have had either i) previous surgery and radiotherpay, ii) adjuvant/neoadjuvant chemotherapy, iii) adjuvant and/or palliative hormonal therapy. Baseline studies may include complete physical examination, chest radiography, computer axial tomography examination of the abdomen, routine blood counts and chemistries.

Dose, Administration and Treatment Schedule

11D10 is provided as an aluminum hydroxide precipitated suspension at a concentration of 2 mg/ml. 3H1 is provided as an aluminum hydroxide precipitated suspension of 2 mg/ml. Patients receive 11D10 and 3H1 every two weeks for 4 doses and then every 28 days thereafter in combination with their first-line hormonal therapy. First-line therapy includes hormonal therapy, such as with Femara, Arimidex, Aromasin or Tamoxifen. In this study, the antibodies are administered as 2 mg intradermal (ID) injections every other week for 4 doses followed by subcutaneous (SC) maintenance injections every 28 days. Hormonal therapy begins within 28 days prior to antibody administration.

Once the first four doses of antibodies have been administered, the schedule of events for antibody administration (biweekly administration for 4 injections, then every other 4 weeks for 19 months) is followed regardless of hormonal therapy frequency, or changes in regimen. 11D10 is generally administered as a 1 ml volume of 11D10 2 mg in preservative-free saline and 3H1 is administered as a 1 ml volume of 3H1 2 mg in preservative-free saline.

Patients continue on 11D10 and 3H1 for up to twenty-one (21) months and are then followed for survival. Patients may continue to receive hormonal therapy or second line therapy as deemed appropriate by their treating physicians. Patients may continue to receive the antibodies after discontinuation of hormonal therapy or second line therapy, in the event of diminished performance status or disease progression, for up to 21 months. Those patients who show complete response (CR), Partial Response (PR) or Stable Disease/No Change (SD) beyond 21 months of trial therapy, may continue with study drug, if requested.

Assays for Immune Response

Blood samples are collected at baseline, week 10, week 14, week 30, week 42, week 54, week 66, week 78 and week 82/discontinuation in order to evaluate immune response and other surrogate markers of clinical benefit. Various assays can be used to determine immune response to administration of 11D10 and 3H1, including assays described in the preceding Examples, and HAMA, binding of Ab3 from patient serum to CEA, peripheral blood mononuclear cell proliferation in response to 3H1, binding of Ab3 from patient serum to HMFG and peripheral blood mononuclear cell proliferation in response to 11D10.

(i) HAMA: This assay measures the level of human anti-mouse antibodies (HAMA), which are endogenous antibodies against mouse immunoglobulin. Since 11D10 and 3H1 are injected as intact murine IgG1, patients are expected to mount human anti-mouse antibody responses.

(ii) Binding of Ab3 from patient serum to CEA: To assess humoral immune responses that are directed against the CEA tumor antigen, patients' whole or partially-purified sera re tested for specific immunoreactivity to purified recombinant CEA coated onto microtiter plates by ELISA or radioimmunoassay (RIA). The specific antigen-antibody (Ab3) complex is detected using enzyme-conjugated anti-human IgG (H+L chain) reagents, or with ¹²⁵I-labeled or enzyme-conjugated 3H1 antibody (Ab2). Patients' pre-immune sera and an unrelated antigen can be used as independent controls for the assay. A standard curve is generated using purified 8019 antibody (Ab1), and the quantity of Ab3 antibody sera is estimated from the standard curve.

(iii) Proliferation in Response to 3H1: Peripheral blood mononuclear cells (PBMC) separated by Ficoll Hypaque gradient centrifugation from venous blood are cryopreserved using a Cryo Med, in 2 ml vials each containing 10⁷ cells. The cells are thawed immediately before the assay, counted in the presence of Trypan Blue dye, and plated in 96-well plates at 1×10⁵ cells/well.

Following the addition of the 3H1 antibody (Ab2) (2-10 μg/well), control isotype-antibody (2-10 μg/well), or PHA-P (1, 2 and 5 μg/well) as positive control, in RPMI supplemented with 10% pooled human AB serum and antibiotics, the plates are incubated for 5 days in a CO₂ incubator. On day 5, the cultures are pulsed with [³H] thymidine (1 μCi/well) for 24 h. The counts/min obtained from triplicate wells are averaged, and the stimulation indices determined for all cultures.

(iv) Binding of Ab3 from patient serum to HMFG: To assess humoral immune responses that are directed against the HMFG tumor antigen, patients' whole or partially-purified sera are tested for specific immunoreactiviy to a preparation of purified HMFG coated onto microtiter plates by ELISA or radioimmunoassay (RIA). The specific antigen-antibody (Ab3) complex is detected using enzyme-conjugated anti-human IgG (H+L chain) reagents, or with ¹²⁵I-labeled or enzyme-conjugated 11D10 antibody (Ab2). Patients' pre-immune sera and an unrelated antigen are used as independent controls for the assay. A standard curve is generated using purified MC-10 (BrE-1) antibody (Ab1), and the quantity of Ab3 antibody sera is estimated from the standard curve.

(v) Proliferation in Response to 11D10: Peripheral blood mononuclear cells (PBMC) separated by Ficoll Hypaque gradient centrifugation from venous blood are cryopreserved using a Cryo Med, in 2 ml vials each containing 10⁷ cells. The cells are thawed immediately before the assay, counted in the presence of Trypan Blue dye, and plated in 96-well plates at 1×10⁵ cells/well. Following the addition of the 11D10 antibody (Ab2) (2-10 μg/well), control isotype-antibody (2-10 μg/well), or PHA-P (1, 2 and 5 μg/well) as positive control, in RPMI supplemented with 10% pooled human AB serum and antibiotics, the plates are incubated for 5 days in CO₂ incubator. On day 5, the cultures are pulsed with [³H] thymidine (1 μCi/well) for 24 h. The counts/min obtained from triplicate wells are averaged, and the stimulation indices determined for all cultures.

Survival Results

Survival data is calculated based on length of time a patient has no detectable disease ( i.e., length of time until progression). This length of time is determined based on entry date into the study. An even more meaningful statistic is length of time of no detectable disease (or to progression) as measured from the date of the last treatment. Disease progression can also be measured based on progression in the disease as indicated by staging markers for the particular cancer type.

### Example 9

### Study of Post-operative Adjuvant Immunotherapy with 11D10 and 3H1 and Radiation

Selection of Patients

Patients with HMFG- and CEA-positive tumors, generally non-small cell lung cancer, are selected for this study. These patients generally have histologic documentation of non-small cell lung cancer (NSCLC). They generally have Stage II and Stage IIIA disease; patients with N1 disease are eligible only if there is pathologic involvement of hilar lymph nodes. Generally, a pathologic diagnosis of Stage II/IIIA has been made at the time of surgical resection. Generally, patients have had surgery (within about 7 weeks prior to study entry) consisting of lobectomy, sleeve resection, bilobectomy or pneumonectomy.

Dose, Administration and Treatment Schedule

2 mg of 3H1 Alu-Gel and 2 mg of 1ID10 Alu-Gel are administered intracutaneously at separate sites once a week for three weeks, starting weeks 2-7 after surgery, then monthly subcutaneously for two years. Antibodies are given on different sites, such as in different arms. Vital signs are obtained every 15 minutes for at least 30 minutes following injections. Vaccines are generally given by day 45 following surgery. Concurrent radiotherapy starts within 1 week following the third 20 weekly post-op vaccination and within 9 weeks after surgery, at 50.4 Gy/28 fractions/5-6 weeks (1.8 Gy/day, 5 days/week) with 10.8 Gy/6 fractions boost to nodal stations if there is extracapsular extension of nodal metastases.

Assays for Immune Response

Serum from treated individuals is tested to evaluate immune response and other surrogate markers of clinical benefit. Serum can be collected at 6, 12, 16, 24, 36 and 48 months during the treatment period. Various assays can be used to determine immune response to administration of 11D10 and 3H1, including assays described in the preceding Examples, and binding of antibody from patient serum to CEA, proliferation in response to 3H1, binding of antibody from patient serum to HMFG and proliferation in response to 11D10.

(i) Binding of Ab3 from patient serum to CEA: To assess humoral immune responses that are directed against the CEA tumor antigen, patients' whole or partially-purified sera are tested for specific immunoreactivity to purified recombinant CEA coated onto microtiter plates by ELISA or radioimmunoassay (RIA). The specific antigen-antibody (Ab3) complex is detected using enzyme-conjugated anti-human IgG (H+L chain) reagents, or with ¹²⁵I-labeled or enzyme-conjugated 3H1 antibody (Ab2). Patients' pre-immune sera and an unrelated antigen can be used as independent controls for the assay. A standard curve is generated using purified 8019 antibody (Ab1), and the quantity of Ab3 antibody sera can be estimated from the standard curve.

(ii) Proliferation in Response to 3H1: Peripheral blood mononuclear cells (PBMC) separated by Ficoll Hypaque gradient centrifugation from venous blood are cryopreserved using a Cryo Med, in 2 ml vials each containing 10⁷ cells. The cells are thawed immediately before the assay, counted in the presence of Trypan Blue dye, and plated in 96-well plates at 1×10⁵ cells/well.

Following the addition of 3H1 antibody (Ab2) (2-10 μg/well), control isotype-antibody (2-10 μg/well), or PHA-P (1, 2 and 5 μg/well) as positive control, in RPMI supplemented with 10% pooled human AB serum and antibiotics, the plates are incubated for 5 days in a CO₂ incubator. On day 5, the cultures are pulsed with [³H] thymidine (1 μCi/well) for 24 h. The counts/min obtained from triplicate wells are averaged, and the stimulation indices determined for all cultures.

(iii) Binding of Ab3 from patient serum to HMFG: To assess humoral immune responses that are directed against the HMFG tumor antigen, patients' whole or partially-purified sera are tested for specific immunoreactiviy to a preparation of purified HMFG coated onto microtiter plates by ELISA or radioimmunoassay (RIA).

The specific antigen-antibody (Ab3) complex is detected using enzyme-conjugated anti-human IgG (H+L chain) reagents, or with ¹²⁵I-labeled or enzyme-conjugated anti-id 11D10 antibody (Ab2). Patients' pre-immune sera and an unrelated antigen can be used as independent controls for the assay. A standard curve is generated using purified MC-10 (BrE-1) antibody (Ab1), and the quantity of Ab3 antibody sera can be estimated from the standard curve.

(iv) Proliferation in Response to 11D10: Peripheral blood mononuclear cells (PBMC) separated by Ficoll Hypaque gradient centrifugation from venous blood are cryopreserved using a Cryo Med, in 2 ml vials each containing 10⁷ cells. The cells are thawed immediately before the assay, counted in the presence of Trypan Blue dye, and plated in 96-well plates at 1×10⁵ cells/well.

Following the addition of the 11D10 antibody (Ab2) (2-10 μg/well), control isotype-antibody (2-10 μg/well), or PHA-P (1, 2 and 5 μg/well) as positive control, in RPMI supplemented with 10% pooled human AB serum and antibiotics, the plates are incubated for 5 days in CO₂ incubator. On day 5, the cultures are pulsed with [³H] thymidine (1 μCi/well) for 24 h. The counts/min obtained from triplicate wells are averaged, and the stimulation indices determined for all cultures.

Survival Results

Data for recurrence, disease-free survival and survival can be collected. Generally, recurrence occurs when there is development of a loco-regional and/or distant recurrence. Generally, disease-free survival is determined based on date of definitive resection to the date of first treatment failure (such as recurrence or death before recurrence). Generally, survival is determined based on the time from definitive resection until death (generally cancer-related death).

Although the foregoing invention has been described in some detail by way of illustration and example for purposes of clarity of understanding, it will be apparent to those skilled in the art that certain changes and modifications may be practiced. Therefore, the descriptions and examples should not be construed as limiting the scope of the invention, which is delineated by the appended claims.

What is claimed is:

1. A method of delaying development of an HMFG- and CEA-associated tumor in an individual, comprising administering an effective amount of a first antibody comprising the light and heavy chain variable region sequences contained in SEQ ID NO:2 and SEQ ID NO:4, respectively, and a second antibody comprising the light and heavy chain variable region sequences contained in SEQ ID NO:6 and SEQ ID NO:8, respectively, to the individual.

2. The method of claim 1, wherein said first antibody is antibody 11D10, which is produced by a hybridoma cell line deposited at the American Type Culture Collection (ATCC) as Accession No. 12020, or progeny thereof.

3. The method of claim 1, wherein said second antibody is antibody 3H1, which is produced by a hybridoma cell line deposited at the American Type Culture Collection (ATCC) as Accession No. 12003, or progeny thereof.

4. The method of claim 1, wherein said first antibody is antibody 11D10, which is produced by a hybridoma cell line deposited at the American Type Culture Collection (ATCC) as Accession No. 12020, or progeny thereof, and the second antibody is antibody 3H1, which is produced by a hybridoma cell line deposited at the American Type Culture Collection (ATCC) as Accession No. 12003, or progeny thereof.

5. The method of claim 1, wherein said individual has a low tumor burden.

6. The method of claim 1, wherein the individual is high risk.

7. The method of claim 6, wherein the individual is in the adjuvant setting.

8. The method of claim 1, wherein the first antibody is administered with an adjuvant.

9. The method of claim 1, wherein the second antibody is administered with an adjuvant.

10. The method of claim 1, wherein the first antibody and second antibody are administered with an adjuvant.

11. The method of any of claims 8-10, wherein the adjuvant is aluminum hydroxide.

12. The method of claim 1, wherein the tumor is of gastrointestinal origin.

13. The method of claim 12, wherein the tumor is colorectal.

14. The method of claim 1, wherein the tumor is lung.

15. The method of claim 14, wherein the tumor is non-small cell lung carcinoma.

16. The method of claim 14, wherein the tumor is small cell lung carcinoma.

17. The method of claim 1, wherein the tumor is ovarian.

18. The method of claim 1, wherein the tumor is breast.

19. The method of claim 1, wherein the first antibody and the second antibody are each administered in an amount of about 1 mg to about 4 mg.

20. The method of claim 19, wherein the first antibody and the second antibody are each administered in an amount of about 2 mg.

21. The method of claim 1, wherein the first antibody and the second antibody are each administered at weekly intervals.

22. The method of claim 1, wherein the first antibody and the second antibody are each administered every two weeks.

23. The method of claim 1, wherein the first antibody and the second antibody are heat-treated prior to administration.

24. The method of claim 1, wherein the individual has a circulating CEA level of less than about 50 ng/ml.

25. A method of treatment of an HMFG- and CEA-associated tumor in an individual comprising administering an effective amount of a first antibody comprising the light and heavy chain variable region sequences contained in SEQ ID NO:2 and SEQ ID NO:4, respectively, and a second antibody comprising the light and heavy chain variable region sequences contained in SEQ ID NO:6 and SEQ ID NO:8, respectively, to the individual.

26. The method of claim 25, wherein said first antibody is antibody 11D10, which is produced by a hybridoma cell line deposited at the American Type Culture Collection (ATCC) as Accession No. 12020, or progeny thereof.

27. The method of claim 25, wherein said second antibody is antibody 3H1, which is produced by a hybridoma cell line deposited at the American Type Culture Collection (ATCC) as Accession No. 12003, or progeny thereof.

28. The method of claim 25, wherein said first antibody is antibody 11D10, which is produced by a hybridoma cell line deposited at the American Type Culture Collection (ATCC) as Accession No. 12020, or progeny thereof, and the second antibody is antibody 3H1, which is produced by a hybridoma cell line deposited at the American Type Culture Collection (ATCC) as Accession No. 12003, or progeny thereof.

29. The method of claim 25, wherein said individual has a low tumor burden.

30. The method of claim 25, wherein the first antibody and second antibody are administered at monthly intervals.

31. The method of claim 25, wherein the individual is high risk.

32. The method of claim 31, wherein the individual is in the adjuvant setting.

33. The method of claim 25, wherein the first antibody is administered with an adjuvant.

34. The method of claim 25, wherein the second antibody is administered with an adjuvant.

35. The method of claim 25, wherein the first antibody and the second antibody are administered with an adjuvant.

36. The method of any of claims 33-35, wherein the adjuvant is aluminum hydroxide.

37. The method of claim 25, wherein the tumor is of gastrointestinal origin.

38. The method of claim 37, wherein the tumor is colorectal.

39. The method of claim 25, wherein the tumor is lung.

40. The method of claim 39, wherein the tumor is non-small cell lung carcinoma.

41. The method of claim 39, wherein the tumor is small cell lung carcinoma.

42. The method of claim 25, wherein the tumor is ovarian.

43. The method of claim 25, wherein the tumor is breast.

44. The method of claim 25, wherein the first antibody and the second antibody are each administered in an amount of about 1 mg to about 4 mg.

45. The method of claim 44, wherein the first antibody and the second antibody are each administered in an amount of about 2 mg.

46. The method of claim 25, wherein the first antibody and the second antibody are each administered at weekly intervals.

47. The method of claim 25, wherein the first antibody and the second antibody are each administered every two weeks.

48. The method of claim 25, wherein the first antibody and the second antibody are each administered at monthly intervals.

49. The method of claim 25, wherein the first antibody and the second antibody are heat-treated prior to administration.

50. The method of claim 25, wherein the individual has a circulating CEA level of less than about 50 ng/ml.

51. The method of claim 1 or 25, wherein said individual is human.

52. A composition comprising a first antibody comprising the light and heavy chain variable region sequences contained in SEQ ID NO:2 and SEQ ID NO:4, respectively, and a second antibody comprising the light and heavy chain variable region sequences contained in SEQ ID NO:6 and SEQ ID NO:8, respectively.

53. The composition of claim 52, wherein said first antibody is antibody 11D10, which is produced by a hybridoma cell line deposited at the American Type Culture Collection (ATCC) as Accession No. 12020, or progeny thereof.

54. The composition of claim 52, wherein said second antibody is antibody 3H1, which is produced by a hybridoma cell line deposited at the American Type Culture Collection (ATCC) as Accession No. 12003, or progeny thereof.

55. The composition of claim 52, wherein said first antibody is antibody 11D10, which is produced by a hybridoma cell line deposited,at the American Type Culture Collection (ATCC) as Accession No. 12020, or progeny thereof, and the second antibody is antibody 3H1, which is produced by a hybridoma cell line deposited at the American Type Culture Collection (ATCC) as Accession No. 12003, or progeny thereof.

56. A method of identifying an individual suitable for the method of claim 1 or 25, said method comprising detecting both HMFG and CEA in or on the cells of the same tumor in an individual, whereby the presence of HMFG and CEA is indicative of an individual suitable for the method of claim 1 or 25.

57. A method of delaying development of an HMFG- and CEA-associated tumor comprising:
(a) identifying a suitable individual according to the method of claim 56; and
(b) administering an effective amount of a first antibody comprising the light and heavy chain variable region sequences contained in SEQ ID NO:2 and SEQ ID NO:4, respectively, and a second antibody comprising the light and heavy chain variable region sequences contained in SEQ ID NO:6 and SEQ ID NO:8, respectively, to the individual.

58. A method of treatment of an HMFG- and CEA-associated tumor comprising:
(a) identifying a suitable individual according to the method of claim 56; and
(b) administering an effective amount of a first antibody comprising the light and heavy chain variable region sequences contained in SEQ ID NO:2 and SEQ ID NO:4, respectively, and a second antibody comprising the light and heavy chain variable region sequences contained in SEQ ID NO:6 and SEQ ID NO:8, respectively, to the individual.


SEQUENCE LISTING (38 sequences)

SEQ ID NO: 1 - DNA, 435 bp, Mus musculus
  CDS (1)...(435)
  sig_peptide (1)...(60)
  mat_peptide (61)...(435)
atgggggcccctgctcagattcttgggttcttgttgctcttgtttccaggtaccagatgtgacatccagatgacccagtctccatcctccttatctgcctctctgggacaaagagtcagtctcacttgtcgggcaagtcaggacattggtattaacttacattggcttcagcaggaaccagatggaactattaaacgcctgatctacgccacatccagtttaggttctggtgtccccaaaaggttcagtggcagtaggtctgggtcagattattctctcaccatcagcagccttgagtctgaagattttgtagcctattactgtctacaatatgctagttctccgtacacgttcggaggggggaccaagctggaaataaaacgggctgatgctgcaccaactgtatccatcttcccaccatccagtaagcttggg

SEQ ID NO: 2 - protein, 145 aa, Mus musculus
  SIGNAL (1)...(20)
MGAPAQILGFLLLLFPGTRCDIQMTQSPSSLSASLGQRVSLTCRASQDIGINLHWLQQEPDGTIKRLIYATSSLGSGVPKRFSGSRSGSDYSLTISSLESEDFVAYYCLQYASSPYTFGGGTKLEIKRADAAPTVSIFPPSSKLG

SEQ ID NO: 3 - DNA, 461 bp, Mus musculus
  CDS (1)...(461)
  sig_peptide (1)...(57)
  mat_peptide (58)...(461)
atggaatgcagctgggtctttctcttcctcctgtcaataactacaggtgtccactcccaggcttatctacagcagtctggggctgagctggtgaggtctggggcctcagtgaagatgtcctgcaaggcttctggctacacattgaccagttacaatatgcactgggtaaagcagacacctggacagggcctggaatggattggaaatatttttcctggaaatggtgatacttactacaatcagaagtttaagggcaaggcctcattgactgcagacacatcctccagcacagcctacatgcagatcagcagcctgacatctgaagactctgcggtctatttctgtgcaagagggaactgggagggtgctctggactactggggtcaaggaacctcagtcaccgtctcctcagccaaaacgacacccccacccgtctatccactggtccctggaagcttggg

SEQ ID NO: 4 - protein, 153 aa, Mus musculus
  SIGNAL (1)...(19)
MECSWVFLFLLSITTGVHSQAYLQQSGAELVRSGASVKMSCKASGYTLTSYNMHWVKQTPGQGLEWIGNIFPGNGDTYYNQKFKGKASLTADTSSSTAYMQISSLTSEDSAVYFCARGNWEGALDYWGQGTSVTVSSAKTTPPPVYPLVPGSL

SEQ ID NO: 5 - protein, 23 aa, Mus musculus
DIQMTQSPSSLSASLGQRVSLTC

SEQ ID NO: 6 - protein, 11 aa, Mus musculus
RASQDIGINLH

SEQ ID NO: 7 - protein, 15 aa, Mus musculus
TLQQEPDGTIKRLIY

SEQ ID NO: 8 - protein, 7 aa, Mus musculus
ATSSLGS

SEQ ID NO: 9 - protein, 33 aa, Mus musculus
GVPKRFSGSRSGSDYSLTISSLESEEDFVAYYC

SEQ ID NO: 10 - protein, 9 aa, Mus musculus
LQYASSPYT

SEQ ID NO: 11 - protein, 10 aa, Mus musculus
FGGGTKLEIK

SEQ ID NO: 12 - protein, 30 aa, Mus musculus
QAYLQQSGAELVRSGASVKMSCKASGYTLT

SEQ ID NO: 13 - protein, 5 aa, Mus musculus
SYNMH

SEQ ID NO: 14 - protein, 14 aa, Mus musculus
WVKQTPGQGLEWIG

SEQ ID NO: 15 - protein, 17 aa, Mus musculus
NIFPGNGDTYYNQKFKG

SEQ ID NO: 16 - protein, 32 aa, Mus musculus
KASLTADTSSSTAYMQISSLTSEDSAVYFCAR

SEQ ID NO: 17 - protein, 9 aa, Mus musculus
GNWEGALDY

SEQ ID NO: 18 - protein, 11 aa, Mus musculus
WGQGTSVTVSS

SEQ ID NO: 19 - DNA, 447 bp, Mus musculus
tcatatggattactagtcgacatggtatccacagctcagttccttggtatcttgttgctctggtttccaggtatcaaatctgacatcaagatgacccagtctccatcttccatgtatggatctctaggagagagagtcacgatcacttgcaaggcgagtcaggacattaatggttatttaaattggttccaacaagaaccagggaaatctcctaagaccctgatctatcgtgcaaatagattgatagatggggtcccatcaaggttcagtggcagtggatctgggcaagtttactctctcaccatcagcagcctggaatatgaagatatgggaacttattattgtctacagtttgatgagtttccgtggatgttcggtggaggcaccaagctggaaatcaaacgggctgatgctgcaccaactgtctccatcttcccaccatccagt

SEQ ID NO: 20 - protein, 23 aa, Mus musculus
DIKMTQSPSSMYASLGERVTITC

SEQ ID NO: 21 - protein, 23 aa, Mus musculus
DIKMTQSPSSMYASLGERVTITC

SEQ ID NO: 22 - protein, 11 aa, Mus musculus
KASQDINGYLN

SEQ ID NO: 23 - protein, 15 aa, Mus musculus
WFQQEPGKSPKTLIY

SEQ ID NO: 24 - protein, 7 aa, Mus musculus
RANRLID

SEQ ID NO: 25 - protein, 32 aa, Mus musculus
GVPSRFSGSGSGQVYSLTISSLEYEDMGTYYC

SEQ ID NO: 26 - protein, 19 aa, Mus musculus
LQFDEFPWMFGGGTKLEIK

SEQ ID NO: 27 - protein, 15 aa, Mus musculus
RADAAPTVSIFPPSS

SEQ ID NO: 28 - DNA, 462 bp, Mus musculus
agtcatatggattgggaattcatggaatggagctgggtcattctcttcctcctgtcaggaactgcaggtgtccactctgaggtccagctgcaacagtctggacctgagctggtgaagcctggagcttcactgaagatttcctgcgaggcttctggttactcactcactgcctacaccatgaactgggtgaagcagagccatggaaagagccttgagtgggttgggctgattaatcctttcagtggtgatactaactacagccagaaattcacgggcaaggccacattaactgtagacaggtcatccagcacagcctacatggagctcctcagtctgacatctgaggactctgcagtctattactgtgtcattactccggttccctactggtacttcgatgtctggggcgcagggaccacggtcaccgtctcctcagccaaaacgacacccccatccgtctat

SEQ ID NO: 29 - protein, 19 aa, Mus musculus
MEWSWVILFLLSGTAGVHS

SEQ ID NO: 30 - protein, 5 aa, Mus musculus
AYTMN

SEQ ID NO: 31 - protein, 5 aa, Mus musculus
AYTMN

SEQ ID NO: 32 - protein, 14 aa, Mus musculus
WVKQSHGKSLEWVG

SEQ ID NO: 33 - protein, 17 aa, Mus musculus
LINPFSGDTNYSQKFTG

SEQ ID NO: 34 - protein, 31 aa, Mus musculus
KATLVDRSSSTAYMELLSLTSEDSAVYYCVI

SEQ ID NO: 35 - protein, 10 aa, Mus musculus
TPVPYWYFDV

SEQ ID NO: 36 - protein, 11 aa, Mus musculus
WGAGTTVTVSS

SEQ ID NO: 37 - protein, 9 aa, Mus musculus
AKTTPPSVY

SEQ ID NO: 38 - protein, 9 aa, Mus musculus
LQFDEFPWM